(19) United States
(12) Patent Application Publication
(10) Pub. No.: US 2024/0398939 A1
(43) Pub. Date: Dec. 5, 2024

(54) ADENOVIRUSES AND METHODS FOR USING ADENOVIRUSES

(71) Applicant: SAGITTARIUS BIO, INC., Thousand Oaks, CA (US)

(72) Inventors: Kayvan NIAZI, Thousand Oaks, CA (US); Shahrooz RABIZADEH, Thousand Oaks, CA (US)

(21) Appl. No.: 18/694,830

(22) PCT Filed: Sep. 23, 2022
(86) PCT No.: PCT/US2022/076951

§ 371 Date: Mar. 22, 2024

Related U.S. Application Data
(60) Provisional application No. 63/261,561, filed on Sep. 23, 2021.

(51) Int. Cl. A61K 39/29; A61P 37/04; C07K 14/005; C12N 15/86

(52) U.S. Cl. CPC A61K 39/292; A61P 37/04; C07K 14/005; C12N 15/86; C12N 2710/10022; C12N 2710/10043; C12N 2710/10052; C12N 2730/10122; C12N 2730/10171

(57) ABSTRACT

The embodiments provided for herein relate to recombinant adenoviruses, wherein the capsid hexon polypeptides of the adenovirus have been modified. Such modifications can comprise the modification of adenovirus strain Ad5 with at least one capsid hexon hypervariable region polypeptide from adenovirus strain Ad57. The embodiments also relate to the modified capsid hexon polypeptides, to nucleic acids encoding the modified capsid hexon polypeptides, and to methods of using the same.

### PRIORITY

The present application claims priority to U.S. Provisional Application No. 63/261,561, filed on Sep. 23, 2021, which is hereby incorporated by reference in its entirety.

### SEQUENCE LISTING

The present application contains a Sequence Listing which has been submitted electronically is XML format and is hereby incorporated by reference in its entirety. The Sequence Listing, created on Sep. 19, 2022, is called “260034.000102 sequence listing XML” and is 23,241 bytes in size.

### FIELD

The present embodiments relate to recombinant adenoviruses, wherein the capsid hexon polypeptides of the adenovirus have been modified. Such modifications comprise the modification of adenovirus strain Ad5 with at least one capsid hexon hypervariable region polypeptide from adenovirus strain Ad57.

### BACKGROUND

Adenoviruses are widely used in the art for the delivery of a wide range of compounds, polynucleotides, and polypeptides to specific cellular targets. A substantial portion of the adenovirus surface icosahedron is made up of a repeating pattern of hexon proteins. Modifications to the hexons can impact adenovirus targeting, neutralization, capacity, and other factors. Although significant progress has been made to use adenoviruses as therapeutics, there is still a need for improved adenoviruses with modifications that affect specificity, cargo capacity as well as other properties. The embodiments provided for herein fulfill these needs as well as others.

### SUMMARY

In some embodiments, a recombinant adenovirus (Ad) is provided. In some embodiments, capsid hexon polypeptides of an Ad strain Ad5 comprise at least one capsid hexon hypervariable region (HVR) polypeptide from Ad strain Ad57. In some embodiments, the capsid hexon polypeptides of the Ad strain Ad5 comprises one or more capsid hexon HVR polypeptides replacements, insertions, and/or deletions from Ad strain Ad57 provided for herein.

In some embodiments, the hexon polypeptide of Ad strain Ad5 comprising the sequence of AATALEINLE (SEQ ID NO: 3) has SEQ ID NO: 3 replaced by a sequence of DDTQVQVAAE (SEQ ID NO: 4). In some embodiments, the hexon polypeptide of Ad strain Ad5 comprises the residues of EQ between residue E at position 156 and residue V at position 157 of SEQ ID NO: 1. In some embodiments, wherein the hexon polypeptide of Ad strain Ad5 does not comprise an insertion, deletion, or substitution at or between residue T at position 166 and residue F at position 169 of SEQ ID NO: 1. In some embodiments, wherein the hexon polypeptide of Ad strain Ad5 comprises an insertion of a polypeptide comprising the sequence of TNGAA (SEQ ID NO: 5) between residue G at position 187 and residue V at position 188 of SEQ ID NO: 1. In some embodiments, the hexon polypeptide of Ad strain Ad5 does not comprise an insertion, deletion, or substitution at or between residue H at position 218 and residue A at position 220 of SEQ ID NO: 1. In some embodiments, the hexon polypeptide of Ad strain Ad5 comprises a deletion of residue K at position 252 of SEQ ID NO: 1. In some embodiments, the hexon polypeptide of Ad strain Ad5 does not comprise an insertion, deletion, or substitution at or between residue F at position 267 and residue S at position 268 of SEQ ID NO: 1. In some embodiments, the hexon polypeptide of Ad strain Ad5 does not comprise an insertion, deletion, or substitution at or between residue G at position 434 and residue Q at position 435 of SEQ ID NO: 1. In some embodiments, the hexon polypeptide of Ad strain Ad5 comprises a deletion of residues QE at positions 435 and 436 of SEQ ID NO: 1. In some embodiments, the hexon polypeptide of Ad strain Ad5 comprises an insertion of a polypeptide comprising the sequence of TT between residue G at position 438 and residue W at position 439 of SEQ ID NO: 1. In some embodiments, the hexon polypeptide of Ad strain Ad5 comprises an insertion of a polypeptide comprising the sequence of GATT (SEQ ID NO: 6) between residue G at position 438 and residue W at position 439 of SEQ ID NO: 1. In some embodiments, the hexon polypeptide of Ad strain Ad5 comprises a deletion of residue E at position 445 of SEQ ID NO: 1. In some embodiments, the hexon polypeptide of Ad strain Ad5 comprising SEQ ID NO: 7 has SEQ ID NO: 7 replaced by SEQ ID NO: 8. In some embodiments, the hexon polypeptide of Ad strain Ad5 comprising SEQ ID NO: 9 has SEQ ID NO: 9 replaced by SEQ ID NO: 10. In some embodiments, the hexon polypeptide of Ad strain Ad5 comprising SEQ ID NO: 11 has SEQ ID NO: 11 replaced by SEQ ID NO: 12. In some embodiments, the hexon polypeptide of Ad strain Ad5 comprising SEQ ID NO: 13 has SEQ ID NO: 13 replaced by SEQ ID NO: 14. In some embodiments, the hexon polypeptide of Ad strain Ad5 comprising SEQ ID NO: 15 has SEQ ID NO: 15 replaced by SEQ ID NO: 16. In some embodiments, the hexon polypeptide of Ad strain Ad5 comprises SEQ ID NOs: 2 or 18.

In some embodiments, a recombinant Ad is provided that comprises a polynucleotide encoding a heterologous protein. In some embodiments, the heterologous protein is a cytokine, an immunoglobulin, an immunomodulatory protein, a viral protein, a patient specific neoepitope, or any combination thereof. In some embodiments, the viral protein is from HBV, HPV, EBV, CMV, HTLV, Polyoma, or any combination thereof. In some embodiments, the immunomodulatory from is the B7 family. In some embodiments, the patient specific neocpitope is a tumor-specific antigen derived from somatic tumor mutation from a patient.

In some embodiments, a recombinant Ad is provided that comprises a targeting moiety. In some embodiments, the targeting moiety is a moiety that targets the Ad to carbohydrates, cell membranes and cells selected from muscle cells, tumors, cancer cells, kidney cells, liver cells, or mucosal cells.

In some embodiments, a recombinant Ad is provided, where the Ad is a replication competent or is a conditionally-replicating Adenovirus (CRAd). In some embodiments, the CRAd comprises a modified E1A gene encoding an E1A polypeptide. In some embodiments, the CRAd exhibits amino acid substitutions in the E1A polypeptide relative to wild-type E1A polypeptide of an Ad strain.

In some embodiments, a nucleic acid molecule encoding capsid hexon polypeptides of an Ad strain Ad5 comprising at least one capsid hexon HVR polypeptide from Ad strain Ad57 is provided. In some embodiments, the capsid hexon polypeptides of the Ad strain Ad5 comprises one or more capsid hexon HVR polypeptides replacements, insertions, and/or deletions from Ad strain Ad57 provided for herein.

In some embodiments, the nucleic acid molecule encoding a hexon polypeptide comprises a sequence of SEQ ID NO: 17.

In some embodiments, a vector comprising any nucleic acid molecule provided for herein is provided. In some embodiments, the vector is an adenoviral vector comprising a transgene encoding, for example, a heterologous polypeptide. In some embodiments, the adenoviral vector further comprises at least one of an E1 deletion, an E3 deletion, and an E4 deletion.

In some embodiments, a recombinant cell comprising any vector provided for herein is provided.

In some embodiments, a method of producing a vector is provided, the method comprising; (a) growing any recombinant cell provided here under conditions for production of the vector; and (b) isolating the vector from the recombinant cell.

In some embodiments, an immunogenic composition comprising any vector provided for herein is provided.

In some embodiments, a method of inducing an immune response in a subject in need thereof is provided, the method comprising administering to the subject an immunogenic composition provided for herein.

In some embodiments, a recombinant Ad strain Ad5 hexon polypeptide comprising at least one capsid HVR polypeptide from Ad strain Ad57 is provided. In some embodiments, the polypeptide comprises one or more capsid hexon HVR polypeptides replacements, insertions, and/or deletions from Ad strain Ad57 provided for herein.

In some embodiments, a virus particle comprising any polypeptide provided for herein is provided. In some embodiments, the virus particle is an adenovirus particle. In some embodiments, the virus particle further comprises a polynucleotide encoding a transgene or a heterologous protein.

In some embodiments, a pharmaceutical composition comprising any polypeptide or particle provided for herein is provided.

In some embodiments, a method of treating a viral infection is provided, the method comprising administering any recombinant adenovirus provided for herein, wherein the recombinant adenovirus expresses a heterologous viral protein. In some embodiments, the heterologous viral protein is from HBV, HPV, EBV, CMV, HTLV, or Polyoma viral protein.

In some embodiments, a method of treating a cancer is provided, the method comprising administering any recombinant adenovirus provided for herein, wherein the recombinant adenovirus expresses a cytokine and/or a tumor antigen.

### BRIEF DESCRIPTION OF THE DRAWINGS

FIG. 1 depicts data showing the liver and spleen were successfully target by two different recombinant viruses, a lightly modified Ad5 (SBI Ad5) and a Ad5 with a modified hexon protein (SynBAd) showed higher effect on the spleen.

FIG. 2 depicts data showing that both SBI Ad5 and SynBAd show good numbers of IFN-γ SFC/106 splenocytes from the liver and spleen, with results differing by method of administration.

FIG. 3 depicts data showing that both viruses show strong CD8 positive percentage when administered with an antigen.

FIG. 4 depicts data showing that the SynBAd virus has lower ALT or AST liver enzymes levels compared to SBI Ad5.

FIG. 5 depicts data showing that both the SBI Ad5 and SynBAd group show and substantial IFN-γ SFC/106 splenocytes.

FIG. 6 depicts data showing there was no corresponding IL-4 activity, showing the that the effect in FIG. 5 is target specific.

FIG. 7 depicts data showing that both the SBI Ad5 and SynBAd groups had similar levels of multimer positive T cell (MHCI) compared to the sham control and antigen plus adjuvant group.

### DETAILED DESCRIPTION

Unless otherwise defined, all technical and scientific terms used herein have the same meaning as commonly understood by one of ordinary skill in the art. Although methods and materials similar or equivalent to those described herein can be used in the practice or testing, suitable methods and materials are described herein. In the case of conflict, the present specification, including definitions, will control. In addition, the materials, methods, and examples are illustrative only and not intended to be limiting. Other features and advantages of the embodiments provided for herein will be apparent from the present detailed description and claims.

The term “about” or “approximately” means plus or minus 10% of the numerical value of the number with which it is being used. Therefore, about 50% means in the range of 45% 55%.

The singular forms “a”, “an”, and “the” include plural reference unless the context clearly dictates otherwise.

The terms “comprise,” “have,” and “include” and their conjugates, as used herein, mean “including but not limited to.” While various compositions, methods, and devices are described in terms of “comprising” various components or steps (interpreted as meaning “including, but not limited to”), the compositions, methods, and devices can also “consist essentially of” or “consist of” the various components and steps.

The terms “substituting,” “substituted,” “mutating,” or “mutated” as used herein refer to altering, deleting, or inserting one or more amino acids or nucleotides in a polypeptide or polynucleotide sequence to generate a variant of that sequence.

The term “variant” as used herein refers to a polypeptide or polynucleotide that differs from a reference polypeptide or a reference polynucleotide by one or more modifications, including substitutions, insertions, or deletions. Variants of the molecules (e.g., nucleic acid molecules and polypeptides) are provided for herein based on either percent identity or percent homology. In some embodiments, the variants can encompass mutations, such as substitutions, insertions, or deletions, that modify the primary structure (sequence) of such molecules without impacting the activity of the polypeptide that is produced or used. For example, the variants of the polypeptides provided for herein can have 1, 2, 3, 4, 5, 6, 7, 8, 9, or 10 substitutions as compared to the reference sequence. These can be point mutations (substitutions), insertions or deletions. For clarity, an insertion or deletion of two or more contiguous amino acid residues is considered a single mutation, but an insertion or deletion of two different amino acid residues that are not contiguous are considered separate mutations.

The term “vector” means a composition of matter that can be used to deliver a cargo to a target, such as a cell, tissue, organ, and the like. In some embodiments, the vector is capable of being duplicated, which can be referred to as a “replicating vector.” In some embodiments, the vector is a non-replicating vector. In some embodiments, the vector is produced in a packaging cell line. In some embodiments, the vector is a viral vector, such as, but not limited to an adenoviral vector. Adenoviral vectors (“adenoviruses”) can be produced to be non-replicating by deleting genes necessary from replication from the adenoviral genome. For example, the E1, E2, E3, or E4 genes can be deleted, either singularly or in combination with one another. To produce the adenoviral particle, the polypeptides produced by these genes are provided by a packaging cell line. Methods of producing adenoviral particles are well known in the art. In some embodiments, the vector can contain elements, such as origins of replication, polyadenylation signal or selection markers that function to facilitate the duplication or maintenance of these polynucleotides in a biological system.

The term “expression vector” means a vector that can be utilized in a biological system, such as, but not limited to, a cell, tissue, or organ, or in a reconstituted biological system to direct the translation of a polypeptide encoded by a polynucleotides sequence present in the expression vector.

The term “polynucleotide” means a molecule comprising a chain of nucleotides covalently linked by a sugar-phosphate backbone or other equivalent covalent chemistry. Double and single-stranded DNAs and RNAs are typical example of polynucleotides.

The term “polypeptide” or “protein” means a molecule that comprises at least two amino acid resides linked by a peptide bond to form a polypeptide. In some embodiments, the term “peptide” can also be used.

As used herein, a “hexon” is an adenovirus protein. Without being bound by any particular theory, an adenovirus icosahedron is typically made up of 240 copies of its hexon protein. The hexon polypeptide comprises a conserved region in its C-terminus and a plurality of hypervariable regions (HVRs) in the N-terminus portion of the protein. Without being bound by any particular theory, the HVRs affect antigenicity of the adenoviral particle as well as the tissue that the adenoviral particle will target. Thus, the tropism of the adenoviral particle can be modified by the formation of a chimeric hexon polypeptide, which can be used to generate an adenoviral particle. In some embodiments, the chimeric hexon polypeptide increases the specificity for the liver. In some embodiments, the chimeric hexon polypeptide is a chimeric as provided for herein, which can be, but is not limited to an Ad5/Ad57 chimeric hexon polypeptide as provided for herein.

In some embodiments, a recombinant adenovirus (Ads) is provided that comprises a chimeric hexon polypeptide. In some embodiments, the chimeric hexon polypeptide comprises a portion that is derived from a first Ad and a portion that is derived from a second Ad. The first portion can, in some embodiments, be referred to as the backbone on to which the portions of the second Ad are grafted on to. In some embodiments, the hexon polypeptide sequences of the second Ad are used to replace a portion of the hexon polypeptide sequence of the first Ad. In some embodiments, the hexon polypeptide sequence of the second Ad is inserted into a region of the first hexon polypeptide sequence. For example, a hexon from a first Ad, such as Ad5, can be modified with one or more HVRs from a second Ad, such as Ad57. The creation of recombinant Ads with modified or chimeric hexons can influence aspects of the Ad, including but not limited to the carrying capacity of the Ad, antibody neutralization rates of the Ad, and Ad targeting. For example, modification of Ad hexon polypeptides can influence adenovirus targeting to various organs, either increasing or decreasing affinity for an organ or tissue. In some embodiments, Ad the hexon modification affects targeting to the liver. In some embodiments, an adenovirus with a chimeric hexon polypeptide comprising a chimeric of an Ad5/Ad57 hexon polypeptide increases transduction of the adenovirus in the liver.

In some embodiments, the recombinant Ads can be used for oncolytic anti-cancer activity. For example, a recombinant Ad can be derived from a first Ad and can include hexon HVRs from one or more different Ads. The HVRs may be derived from any species C Ads, for example Ad1, Ad2, Ad5, Ad6 and Ad57. In some embodiments, a recombinant Ad can be derived from a first Ad and can include one or more hexon HVRs from at least one other Ad, wherein at least one hexon HVR is different from the HVR(s) of the first Ad. In some embodiments, the first Ad strain can be a human Ad5 strain, and the second Ad strain can be a human Ad57 strain.

In some embodiments, recombinant Ads described herein can be replication competent Ads (RC-Ads). For example, a RC-Ad can be a RC-Ad that includes a nucleic acid encoding an E1 polypeptide (e.g., an E1+RC-Ad). For example, a RC-Ad can be a single-cycle Ad (SC-Ad) that includes a deletion of one or more nucleic acids encoding one or more polypeptides associated with the production of infectious viral progeny (e.g., pIIIa and E3). For example, a RC-Ad can be a conditionally-replicating Ad (CRAd). The replication competent adenoviruses that can comprise the chimeric hexon polypeptides provided for herein can be any replication competent adenovirus. Examples of such replication competent adenovirus particles are described in, but not limited to, US 2021/0017501, US 2020/0397839, US 2019/0076493, US 2019/0055522, US 2018/0311291, US 2018/0169271, US20150231229, and US20120283318, each of which is hereby incorporated by reference in its entirety. These are merely non-limiting examples and any replication competent adenovirus can also be used.

Nucleic acid and/or polypeptides that do not naturally occur in the Ad can be from any appropriate source. In some embodiments, a nucleic acid and/or a polypeptide that does not naturally occur in that Ad can be from a non-viral organism. In some embodiments, a nucleic acid and/or a polypeptide that does not naturally occur in that Ad can be from a virus other than an Ad. In some embodiments, a nucleic acid and/or a polypeptide that does not naturally occur in that Ad can be from an Ad obtained from a different species. In some embodiments, a nucleic acid and/or a polypeptide that does not naturally occur in that Ad can be from a different strain of Ad (e.g., serotypically distinct strains). In some embodiments, a nucleic acid and/or a polypeptide that does not naturally occur in that Ad can be a synthetic nucleic acid and/or a synthetic polypeptide.

In some embodiments, a recombinant Ad described herein can include an Ad genome containing one or more substitutions. For example, one or more nucleic acids encoding a polypeptide (or fragments thereof) and/or one or more viral elements encoded by the Ad genome can be substituted. A substitution can be any appropriate substitution. In some embodiments, one or more nucleic acids encoding a capsid polypeptide of a genome of a first Ad can be substituted with one or more nucleic acids encoding a capsid polypeptide of a second Ad to generate a chimeric Ad. For example, when a recombinant Ad includes a genome from a first Ad where a nucleic acid encoding a capsid polypeptide in the genome is substituted for a nucleic acid encoding a capsid polypeptide from a second Ad (e.g., an Ad different from the Ad backbone), the nucleic acid encoding a capsid polypeptide form the second Ad can express one or more capsid polypeptides, and the expressed capsid polypeptide(s) can be incorporated into the capsid of the recombinant Ad. Examples of capsid polypeptides include, without limitation, hexon polypeptides, fiber polypeptides, penton base polypeptides, IIIa polypeptides, IX polypeptides, and pVI polypeptides.

In some embodiments, a recombinant Ad can include a genome from a first Ad where a nucleic acid encoding a hexon polypeptide (e.g., HVRs of a nucleic acid encoding a hexon polypeptide) in the genome is substituted for a nucleic acid encoding a hexon polypeptide (e.g., HVRs of a nucleic acid encoding a hexon polypeptide) from a second Ad. In some embodiments, a recombinant Ad described herein can include a genome from a first Ad that has one or more HVRs substituted for one or more HVRs from a second Ad. In some embodiments, where a recombinant Ad includes a genome from a first Ad where a nucleic acid encoding a hexon polypeptide in the genome is substituted for a nucleic acid encoding a hexon polypeptide from a second Ad, the recombinant Ad can include from about 1 to about 720 hexon polypeptides from the second Ad.

In some embodiments, a recombinant Ad described herein can include an Ad genome containing one or more nucleic acid deletions. A nucleic acid deletion can be any appropriate nucleic acid deletion. A nucleic acid deletion can be a full deletion (e.g., deletion of a nucleic acid encoding a polypeptide) or a partial deletion (e.g., deletion of one or more nucleotides within a nucleic acid encoding a polypeptide). A nucleic acid deletion can reduce or eliminate transcription and translation of a polypeptide encoded by the deleted nucleic acid. Any appropriate nucleic acid can be deleted. In some embodiments, a nucleic acid encoding a polypeptide associated with production of infectious progeny can be deleted. Examples of nucleic acids that can be deleted and/or modified in a recombinant Ad described herein may encode E1 (e.g., E1A and E1B), E2, E3, E4, pIIIA, fiber, E1B, and include viral enhancers and promoters. For example, a recombinant Ad described herein can include an Ad genome containing a deletion of one or more nucleotides within a nucleic acid encoding an E1 polypeptide. In some embodiments, a recombinant Ad described herein can include one or more substitutions in a nucleic acid encoding an E1 polypeptide. The proteins encoded by the genes or nucleic acid molecules that are deleted can be supplied back to form the adenoviral particle through the use of a packaging cell line. The use of packaging cell lines to produce adenoviral particles is well known in the art.

In some embodiments, a recombinant Ad described herein can include an Ad genome containing one or more nucleic acid insertions. For example, a nucleic acid insertion can include a nucleic acid encoding a polypeptide. A nucleic acid can be inserted into any appropriate location within a genome of a recombinant Ad described herein. In some embodiments, a nucleic acid encoding a polypeptide can be inserted into a HVR (e.g., HVR 5 loop) of a genome of a recombinant Ad described herein. For example, when a nucleic acid encoding a polypeptide is inserted into a HVR of a genome of a recombinant Ad described herein, the nucleic acid encoding a polypeptide can express one or more polypeptides, and the expressed polypeptide(s) can be incorporated into the capsid of the recombinant Ad. In cases where a nucleic acid encoding a polypeptide is inserted into a HVR of a genome of a recombinant Ad described herein, the recombinant Ad can present from about 1 to about 720 polypeptides encoded by the inserted nucleic acid on its surface. A nucleic acid insertion can be nucleic acid encoding any appropriate polypeptide. In some embodiments, a nucleic acid insertion can encode a polypeptide antigen.

Any of the recombinant Ads or polypeptides described herein may be modified with conservative amino acid substitutions. A “conservative amino acid substitution” is one in which the amino acid residue is replaced with an amino acid residue having a similar side chain. Families of amino acid residues having similar side chains have been defined in the art. For example, these families include amino acids with basic side chains (e.g., K, R, H), acidic side chains (e.g., D, E), uncharged polar side chains (e.g., G, N, Q, S, T, Y, C, H), nonpolar side chains (e.g., G, A, V, L, I, P, F, M, W), beta-branched side chains (e.g., T, V, I) and aromatic side chains (e.g., Y, F, W, H). Thus, in some embodiments, a recombinant Ad or polypeptide, for example, may be replaced with another amino acid residue from the same side chain family. Other examples of acceptable substitutions are substitutions based on isosteric considerations (e.g. norleucine for methionine) or other properties (e.g. 2-thienylalanine for phenylalanine).

Also provided herein are expression vectors encoding for a recombinant Ad provided for herein. Expression vectors can comprise nucleic acid molecules encoding for a recombinant Ad described herein into another cell to produce the recombinant Ad, wherein the Ad particle can be produced. In some embodiments, an expression vector, which can also be referred to as an expression construct, can be, for example, a plasmid or other type of vector (e.g. virus, liner DNA, and the like) having an enhancer/promoter region controlling expression of one or more nucleic acid molecules. When introduced into a cell, the expression vector can, for example, use the cellular machinery to produce the virus from the cell. In some embodiments, expression vectors containing recombinant Ads described herein can be viral vectors. For example, an expression vector encoding for a recombinant Ad described herein can be a retroviral vector. In some embodiments, expression vectors encoding for a recombinant Ad described herein also can be designed to allow insertion of one or more transgenes (e.g., at a multi-cloning site). For example, expression vectors encoding for a recombinant Ad described herein also can include a nucleic acid encoding any heterologous gene or protein. Examples of such heterologous genes or proteins can be detectable labels, tumor specific antigens, therapeutic proteins, or any desired or chosen heterologous protein. Examples of detectable labels include, without limitation, fluorophores (e.g., green fluorescent protein (GFP), mCherry, and mBFP), and enzymes (e.g., luciferase, recombinases, nucleases, and transcription factors). In some embodiments, the heterologous protein is a plurality of proteins encoded by the transgene. For example, the transgene that is carried by the adenoviral vector can be one or more tumor antigens. In some embodiments, the transgene encodes a general neoantigen (viral; eg HBV polytope), a patient specific neoepitope, or another unrelated target protein from another virus. The transgene can be used to encode any protein, RNA, miRNA, siRNA, CRNA, or molecule that can be encoded by the transgene nucleic acid insert.

In some embodiments, methods and materials for using one or more recombinant Ads or polypeptides described herein are provided. In some embodiments, a recombinant Ad or polypeptide provided herein can used for treating a mammal having, or at risk of having, cancer or viral infection. For example, methods for treating a mammal having, or at risk of having, cancer can include administering one or more recombinant Ads or polypeptides described herein to the mammal. In some embodiments, methods for treating a mammal having, or at risk of having, cancer can include administering one or more expression vectors that encode a recombinant Ad or polypeptide described herein or nucleic acid encoding a recombinant Ad or polypeptide described herein to the mammal. In some embodiments, one or more recombinant Ads or polypeptides described herein can be administered to a mammal to reduce the number of cancer cells in the mammal (e.g., suppress and/or delay tumor growth). In some embodiments, one or more recombinant Ads or polypeptides described herein can be administered to a mammal to reduce the viral titer of an infectious agent in the subject.

### Recombinant Adenoviruses

In some embodiments, recombinant adenoviruses are provided for herein. In some embodiments, a recombinant adenovirus (Ad) comprises a capsid hexon polypeptides of an Ad strain Ad5 comprising one more HVRs of AD57.

In some embodiments, a capsid hexon polypeptide of Ad5 has the following amino acid sequence:

(SEQ ID NO: 1)
MATPSMMPQWSYMHISGQDASEYLSPGLVQFARATETYFSLNNK
FRNPTVAPTHDVTTDRSQRLTLRFIPVDREDTAYSYKARFTLAV
GDNRVLDMASTYFDIRGVLDRGPTFKPYSGTAYNALAPKGAPNP
CEWDEAATALEINLEEEDDDNEDEVDEQAEQQKTHVFGQAPYSG
INITKEGIQIGVEGQTPKYADKTFQPEPQIGESQWYETEINHAA
GRVLKKTTPMKPCYGSYAKPTNENGGQGILVKQQNGKLESQVEM
QFFSTTEATAGNGDNLTPKVVLYSEDVDIETPDTHISYMPTIKE
GNSRELMGQQSMPNRPNYIAFRDNFIGLMYYNSTGNMGVLAGQA
SQLNAVVDLQDRNTELSYQLLLDSIGDRTRYFSMWNQAVDSYDP
DVRIIENHGTEDELPNYCFPLGGVINTETLTKVKPKTGQENGWE
KDATEFSDKNEIRVGNNFAMEINLNANLWRNFLYSNIALYLPDK
LKYSPSNVKISDNPNTYDYMNKRVVAPGLVDCYINLGARWSLDY
MDNVNPFNHHRNAGLRYRSMLLGNGRYVPFHIQVPQKFFAIKNL
LLLPGSYTYEWNFRKDVNMVLQSSLGNDLRVDGASIKFDSICLY
ATFFPMAHNTASTLEAMLRNDTNDQSFNDYLSAANMLYPIPANA
TNVPISIPSRNWAAFRGWAFTRLKTKETPSLGSGYDPYYTYSGS
IPYLDGTFYLNHTFKKVAITFDSSVSWPGNDRLLTPNEFEIKRS
VDGEGYNVAQCNMTKDWFLVQMLANYNIGYQGFYIPESYKDRMY
SFFRNFQPMSRQVVDDTKYKDYQQVGILHQHNNSGFVGYLAPTM
REGQAYPANFPYPLIGKTAVDSITQKKFLCDRTLWRIPFSSNFM
SMGALTDLGQNLLYANSAHALDMTFEVDPMDEPTLLYVLFEVFD
VVRVHRPHRGVIETVYLRTPFSAGNATT

In some embodiments, the capsid hexon polypeptides of an Ad strain Ad5 comprise at least one capsid hexon hypervariable region (HVR) polypeptide from Ad strain Ad57. In some embodiments, a capsid hexon polypeptide of Ad57 has the following amino acid sequence:

(SEQ ID NO: 2)
MATPSMMPQWSYMHISGQDASEYLSPGLVQFARATETYFSLNNK
FRNPTVAPTHDVTTDRSQRLTLRFIPVDREDTAYSYKARFTLAV
GDNRVLDMASTYFDIRGVLDRGPTFKPYSGTAYNALAPKGAPNS
CEWDEDDTQVQVAAEDDQDDDEEEEQLPQQRNGKKTHVYAQAPF
AGEAINKNGLQIGTNGAATEGNKEIYADKTYQPEPQIGESQWNE
AESSVAGGRVLKKTTPMKPCYGSYARPTNSNGGQGVMVEQNGKL
ESQVEMQFFSTSVNAMNEANAIQPKLVLYSEDVNMETPDTHLSY
KPGKSDDNSKAMLGQQSMPNRPNYIAFRDNFIGLMYYNSTGNMG
VLAGQASQLNAVVDLQDRNTELSYQLLLDSIGDRTRYFSMWNQA
VDSYDPDVRIIENHGTEDELPNYCFPLGGIGVTDTYQAIKATNG
NGGATTWAQDNTFAERNEIGVGNNFAMEINLNANLWRNFLYSNI
ALYLPDKLKYNPTNVEISDNPNTYDYMNKRVVAPGLVDCYINLG
ARWSLDYMDNVNPFNHHRNAGLRYRSMLLGNGRYVPFHIQVPQK
FFAIKNLLLLPGSYTYEWNFRKDVNMVLQSSLGNDLRVDGASIK
FDSICLYATFFPMAHNTASTLEAMLRNDTNDQSFNDYLSAANML
YPIPANATNVPISIPSRNWAAFRGWAFTRLKTKETPSLGSGYDP
YYTYSGSIPYLDGTFYLNHTFKKVAITFDSSVSWPGNDRLLTPN
EFEIKRSVDGEGYNVAQCNMTKDWFLVQMLANYNIGYQGFYIPE
SYKDRMYSFFRNFQPMSRQVVDDTKYKDYQQVGILHQHNNSGFV
GYLAPTMREGQAYPANFPYPLIGKTAVNSITQKKFLCDRTLWRI
PFSSNFMSMGALTDLGQNLLYANSAHALDMTFEVDPMDEPTLLY
VLFEVFDVVRVHQPHRGVIETVYLRTPFSAGNATT

In some embodiments, the hexon polypeptide of Ad strain Ad5 comprising the sequence of AATALEINLE (SEQ ID NO: 3) has SEQ ID NO: 3 replaced by a sequence of DDTQVQVAAE (SEQ ID NO: 4).

In some embodiments, the hexon polypeptide of Ad strain Ad5 comprises the residues of EQ between residue E at position 156 and residue V at position 157 of SEQ ID NO: 1.

In some embodiments, the hexon polypeptide of Ad strain Ad5 does not comprise an insertion, deletion, or substitution at or between residue T at position 166 and residue F at position 169 of SEQ ID NO: 1.

In some embodiments, the hexon polypeptide of Ad strain Ad5 comprises an insertion of a polypeptide comprising the sequence of TNGAA (SEQ ID NO: 5) between residue G at position 187 and residue V at position 188 of SEQ ID NO: 1.

In some embodiments, the hexon polypeptide of Ad strain Ad5 does not comprise an insertion, deletion, or substitution at or between residue H at position 218 and residue A at position 220 of SEQ ID NO: 1.

In some embodiments, the hexon polypeptide of Ad strain Ad5 comprises a deletion of residue K at position 252 of SEQ ID NO: 1.

In some embodiments, the hexon polypeptide of Ad strain Ad5 does not comprise an insertion, deletion, or substitution at or between residue F at position 267 and residue S at position 268 of SEQ ID NO: 1.

In some embodiments, the hexon polypeptide of Ad strain Ad5 does not comprise an insertion, deletion, or substitution at or between residue G at position 434 and residue Q at position 435 of SEQ ID NO: 1.

In some embodiments, the hexon polypeptide of Ad strain Ad5 comprises a deletion of residues QE at positions 435 and 436 of SEQ ID NO: 1.

In some embodiments, the hexon polypeptide of Ad strain Ad5 comprises an insertion of a polypeptide comprising the sequence of TT between residue G at position 438 and residue W at position 439 of SEQ ID NO: 1.

In some embodiments, the hexon polypeptide of Ad strain Ad5 comprises an insertion of a polypeptide comprising the sequence of GATT (SEQ ID NO: 6) between residue G at position 438 and residue W at position 439 of SEQ ID NO: 1.

In some embodiments, the hexon polypeptide of Ad strain Ad5 comprises a deletion of residue E at position 445 of SEQ ID NO: 1.

In some embodiments, the hexon polypeptide of Ad strain Ad5 comprising SEQ ID NO: 7 has SEQ ID NO: 7 replaced by SEQ ID NO: 8.

(SEQ ID NO: 7)
PCEWDEAATALEINLEEEDDDNEDEVDEQAEQQKTHVFGQAPYS
GINITKEGIQIGVEGQTPKYADKTFQPEPQIGESQWYETEINHA
AGRVLKKTTPMKPCYGSYAKPTNENGGQGILVKQ
(SEQ ID NO: 8)
SCEWDEDDTQVQVAAEDDQDDDEEEEQLPQQRNGKKTHVYAQAP
FAGEAINKNGLQIGTNGAATEGNKEIYADKTYQPEPQIGESQWN
EAESSVAGGRVLKKTTPMKPCYGSYARPTNSNGGQGVMVE

In some embodiments, the hexon polypeptide of Ad strain Ad5 comprising SEQ ID NO: 9 has SEQ ID NO: 9 replaced by SEQ ID NO: 10.

(SEQ ID NO: 9)
TEATAGNGDNLTPKVVLYSEDVDIETPDTHISYMPTIKEGNSRE
LM
(SEQ ID NO: 10)
SVNAMNEANAIQPKLVLYSEDVNMETPDTHLSYKPGKSDDNSKA
ML

In some embodiments, the hexon polypeptide of Ad strain Ad5 comprising SEQ ID NO: 11 has SEQ ID NO: 11 replaced by SEQ ID NO: 12.

(SEQ ID NO: 11)
VINTETLTKVKPKTGQENGWEKDATEFSDKNEIR
(SEQ ID NO: 12)
IGVTDTYQAIKATNGNGGATTWAQDNTFAERNEIG

In some embodiments, the hexon polypeptide of Ad strain Ad5 comprising SEQ ID NO: 13 has SEQ ID NO: 13 replaced by SEQ ID NO: 14.
SPSNVK (SEQ ID NO: 13)
NPTNVE (SEQ ID NO: 14)

In some embodiments, the hexon polypeptide of Ad strain Ad5 comprising SEQ ID NO: 15 has SEQ ID NO: 15 replaced by SEQ ID NO: 16.

(SEQ ID NO: 15)
PCEWDEAATALEINLEEEDDDNEDEVDEQAEQQKTHVFGQAPYS
GINITKEGIQIGVEGQTPKYADKTFQPEPQIGESQWYETEINHA
AGRVLKKTTPMKPCYGSYAKPTNENGGQGILVKQQNGKLESQVE
MQFFSTTEATAGNGDNLTPKVVLYSEDVDIETPDTHISYMPTIK
EGNSRELMGQQSMPNRPNYIAFRDNFIGLMYYNSTGNMGVLAGQ
ASQLNAVVDLQDRNTELSYQLLLDSIGDRTRYFSMWNQAVDSYD
PDVRIIENHGTEDELPNYCFPLGGVINTETLTKVKPKTGQENGW
EKDATEFSDKNEIRVGNNFAMEINLNANLWRNFLYSNIALYLPD
KLKYSPSNVKISDNPNT
(SEQ ID NO: 16)
SCEWDEDDTQVQVAAEDDQDDDEEEEQLPQQRNGKKTHVYAQAP
FAGEAINKNGLQIGTNGAATEGNKEIYADKTYQPEPQIGESQWN
EAESSVAGGRVLKKTTPMKPCYGSYARPTNSNGGQGVMVEQNGK
LESQVEMQFFSTSVNAMNEANAIQPKLVLYSEDVNMETPDTHLS
YKPGKSDDNSKAMLGQQSMPNRPNYIAFRDNFIGLMYYNSTGNM
GVLAGQASQLNAVVDLQDRNTELSYQLLLDSIGDRTRYFSMWNQ
AVDSYDPDVRIIENHGTEDELPNYCFPLGGIGVTDTYQAIKATN
GNGGATTWAQDNTFAERNEIGVGNNFAMEINLNANLWRNFLYSN
IALYLPDKLKYNPTNVEISDNPNTY

In some embodiments, the chimeric hexon polypeptide of Ad strain Ad5 comprises SEQ ID NO: 2, except that the residue at position 864 is D and/or the residue at position 937 is R as compared to SEQ ID NO: 2. In some embodiments, the chimeric hexon polypeptide of Ad strain Ad5 comprises SEQ ID NO: 2, except that the residue at position 864 is D and the residue at position 937 is R as compared to SEQ ID NO: 2.

In some embodiments, the hexon polypeptide of Ad strain Ad5 comprises SEQ ID NO: 18.

(SEQ ID NO: 18)
MATPSMMPQWSYMHISGQDASEYLSPGLVQFARATETYFSLNNK
FRNPTVAPTHDVTTDRSQRLTLRFIPVDREDTAYSYKARFTLAV
GDNRVLDMASTYFDIRGVLDRGPTFKPYSGTAYNALAPKGAPNS
CEWDEDDTQVQVAAEDDQDDDEEEEQLPQQRNGKKTHVYAQAPF
AGEAINKNGLQIGTNGAATEGNKEIYADKTYQPEPQIGESQWNE
AESSVAGGRVLKKTTPMKPCYGSYARPTNSNGGQGVMVEQNGKL
ESQVEMQFFSTSVNAMNEANAIQPKLVLYSEDVNMETPDTHLSY
KPGKSDDNSKAMLGQQSMPNRPNYIAFRDNFIGLMYYNSTGNMG
VLAGQASQLNAVVDLQDRNTELSYQLLLDSIGDRTRYFSMWNQA
VDSYDPDVRIIENHGTEDELPNYCFPLGGIGVTDTYQAIKATNG
NGGATTWAQDNTFAERNEIGVGNNFAMEINLNANLWRNFLYSNI
ALYLPDKLKYNPTNVEISDNPNTYDYMNKRVVAPGLVDCYINLG
ARWSLDYMDNVNPFNHHRNAGLRYRSMLLGNGRYVPFHIQVPQK
FFAIKNLLLLPGSYTYEWNFRKDVNMVLQSSLGNDLRVDGASIK
FDSICLYATFFPMAHNTASTLEAMLRNDTNDQSFNDYLSAANML
YPIPANATNVPISIPSRNWAAFRGWAFTRLKTKETPSLGSGYDP
YYTYSGSIPYLDGTFYLNHTFKKVAITFDSSVSWPGNDRLLTPN
EFEIKRSVDGEGYNVAQCNMTKDWFLVQMLANYNIGYQGFYIPE
SYKDRMYSFFRNFQPMSRQVVDDTKYKDYQQVGILHQHNNSGFV
GYLAPTMREGQAYPANFPYPLIGKTAVDSITQKKFLCDRTLWRI
PFSSNFMSMGALTDLGQNLLYANSAHALDMTFEVDPMDEPTLLY
VLFEVFDVVRVHRPHRGVIETVYLRTPFSAGNATT

In some embodiments, the adenovirus comprises a hexon polypeptide of SEQ ID NO: 18. In some embodiments, the adenovirus comprises a hexon polypeptide of SEQ ID NO: 2, except that the residue at position 864 is D and/or the residue at position 937 is R as compared to SEQ ID NO: 2. In some embodiments, the adenovirus comprises a hexon polypeptide of SEQ ID NO: 2, except that the residue at position 864 is D and the residue at position 937 is R as compared to SEQ ID NO: 2.

In some embodiments, the capsid hexon polypeptides of the Ad strain Ad5 comprises one or more capsid hexon HVR polypeptides from Ad strain Ad57 provided for herein. In some embodiments, the capsid hexon polypeptides of the Ad strain Ad5 comprises one or more capsid hexon HVR polypeptides from Ad strain Ad57, or any of the insertions or deletions provided for herein. In some embodiments, SEQ ID NO: 1 or any Ad5 hexon sequence known in the art can be modified by the replacement of one or more amino acid sequences by of one or more of SEQ ID NOs: 4, 8, 10, 12, 14, or 16 as described herein; the insertion of one or more of: sequence EQ between SEQ ID NO: 1 position 156 and 157, SEQ ID NO: 5 between SEQ ID NO: 1 position 187 and 188, sequence TT between SEQ ID NO: 1 position 438 and 439, sequence GATT between SEQ ID NO: 1 position 438 and 439; the deletion of one or more of: amino acid K at SEQ ID NO: 1 position 252, amino acids QE at SEQ ID NO: 1 positions 435 and 436, and amino acid E at SEQ ID NO: 1 position 445; and/or any combination of replacement, insertion, or deletion provided for herein.

In some embodiments, any capsid hexon polypeptide of the Ad strain Ad5 provided for herein does not comprise an insertion, deletion, or substitution at or between residue T at position 166 and residue F at position 169 of SEQ ID NO: 1; residue H at position 218 and residue A at position 220 of SEQ ID NO: 1; residue F at position 267 and residue S at position 268 of SEQ ID NO: 1; residue G at position 434 and residue Q at position 435 of SEQ ID NO: 1; and/or any combination thereof.

In some embodiments, any capsid hexon polypeptide of the Ad strain Ad5 provided for herein may be modified by the replacement of any amino acid residue with a conservative amino acid residue of the same type. In some embodiments, any capsid hexon polypeptide of the Ad strain Ad5 provided for herein may be modified by having 1, 2, 3, 4, 5, 6, 7, 8, 9, or 10 amino acids replaced with different amino acids. In some embodiments, a hexon polypeptide of the Ad strain Ad5 is at least 99% homologous or identical to any amino acid sequence or combination of amino acid sequences presented herein. In some embodiments, a hexon polypeptide of the Ad strain Ad5 is at least 98% homologous or identical to any amino acid sequence or combination of amino acid sequences presented herein. In some embodiments, a hexon polypeptide of the Ad strain Ad5 is at least 97% homologous or identical to any amino acid sequence or combination of amino acid sequences presented herein. In some embodiments, a hexon polypeptide of the Ad strain Ad5 is at least 96% homologous or identical to any amino acid sequence or combination of amino acid sequences presented herein. In some embodiments, a hexon polypeptide of the Ad strain Ad5 is at least 95% homologous or identical to any amino acid sequence or combination of amino acid sequences presented herein. In some embodiments, a hexon polypeptide of the Ad strain Ad5 is at least 94% homologous or identical to any amino acid sequence or combination of amino acid sequences presented herein. In some embodiments, a hexon polypeptide of the Ad strain Ad5 is at least 93% homologous or identical to any amino acid sequence or combination of amino acid sequences presented herein. In some embodiments, a hexon polypeptide of the Ad strain Ad5 is at least 92% homologous or identical to any amino acid sequence or combination of amino acid sequences presented herein. In some embodiments, a hexon polypeptide of the Ad strain Ad5 is at least 91% homologous or identical to any amino acid sequence or combination of amino acid sequences presented herein. In some embodiments, a hexon polypeptide of the Ad strain Ad5 is at least 90% homologous or identical to any amino acid sequence or combination of amino acid sequences presented herein. In some embodiments, a hexon polypeptide of the Ad strain Ad5 is at least 85% homologous or identical to any amino acid sequence or combination of amino acid sequences presented herein. In some embodiments, a hexon polypeptide of the Ad strain Ad5 is at least 80% homologous or identical to any amino acid sequence or combination of amino acid sequences presented herein. In some embodiments, a hexon polypeptide of the Ad strain Ad5 is at least 75% homologous or identical to any amino acid sequence or combination of amino acid sequences presented herein. In some embodiments, a hexon polypeptide of the Ad strain Ad5 is at least 70% homologous or identical to any amino acid sequence or combination of amino acid sequences presented herein.

The percent identity of two amino acid or two nucleic acid sequences can be determined by visual inspection and mathematical calculation, or for example, the comparison is done by comparing sequence information using a computer program. An exemplary computer program is the Genetics Computer Group (GCG; Madison, Wis.) Wisconsin package version 10.0 program, GAP (Devereux et al. (1984), Nucleic Acids Res. 12:387-95). The preferred default parameters for the GAP program includes: (1) The GCG implementation of a unary comparison matrix (containing a value of 1 for identities and 0 for non-identities) for nucleotides, and the weighted amino acid comparison matrix of Gribskov and Burgess, ((1986) Nucleic Acids Res. 14:6745) as described in Atlas of Polypeptide Sequence and Structure, Schwartz and Dayhoff, eds., National Biomedical Research Foundation, pp. 353-358 (1979) or other comparable comparison matrices; (2) a penalty of 8 for each gap and an additional penalty of 2 for each symbol in each gap for amino acid sequences, or a penalty of 50 for each gap and an additional penalty of 3 for each symbol in each gap for nucleotide sequences; (3) no penalty for end gaps; and (4) no maximum penalty for long gaps. Other programs used by those skilled in the art of sequence comparison can also be used. In some embodiments, any of the recombinant Ads provided for herein further comprise a polynucleotide encoding a heterologous protein. It is known in the art that a wide variety of polynucleotides, encoding a wide variety of proteins, can be packaged or delivered within Ads. In some embodiments, the heterologous protein is a cytokine, an immunoglobulin, an immunomodulatory protein, a viral protein, a patient specific neoepitope, or any combination thereof, all of which are generally known in the art. In some embodiments, the viral protein is from HBV, HPV, EBV, CMV, HTLV, Polyoma, or any combination thereof. In some embodiments, the immunomodulatory protein is from the B7 family. In some embodiments, the immunomodulatory protein is a B7/Fc, B7.1 or B7.2 protein. In some embodiments, the immunomodulatory protein from the B7 family is fused to a domain from an immunoglobulin protein. In some embodiments, the patient specific neoepitope is a tumor-specific antigen derived from somatic tumor mutation from a patient.

In some embodiments, any of the recombinant Ads provided for herein further comprises a targeting moiety. In some embodiments, the targeting moiety is a moiety that targets the Ad to carbohydrates, cell membranes and cells selected from muscle cells, tumors, cancer cells, kidney cells, liver cells, or mucosal cells.

In some embodiments, any of the recombinant Ads provided for herein are replication competent or is a conditionally-replicating Adenovirus (CRAd) that comprises a modified E1A gene encoding an E1A polypeptide, wherein the CRAd exhibits amino acid substitutions in the E1A polypeptide relative to wild-type E1A polypeptide of an Ad strain. Non-limiting examples of such replication competent adenoviruses are provided for herein.

In some embodiments, a nucleic acid molecule encoding capsid hexon polypeptides of an Ad strain Ad5 is provided. In some embodiments, the nucleic acid molecule encoding capsid hexon polypeptides of an Ad strain Ad5 comprises at least one capsid hexon HVR polypeptide from Ad strain Ad57. In some embodiments, the at least one capsid hexon polypeptides of the Ad strain Ad5 comprises one or more capsid hexon HVR polypeptides from Ad strain Ad57 provided for herein. In some embodiments, the capsid hexon polypeptides of the Ad strain Ad5 comprises one or more capsid hexon HVR polypeptides from Ad strain Ad57, or any of the insertions or deletions provided for herein. In some embodiments, SEQ ID NO: 1 or any Ad5 hexon sequence known in the art can be modified by the replacement of one or more amino acid sequences by of one or more of SEQ ID NOs: 4, 8, 10, 12, 14, or 16 as described herein; the insertion of one or more of: sequence EQ between SEQ ID NO: 1 position 156 and 157, SEQ ID NO: 5 between SEQ ID NO: 1 position 187 and 188, sequence TT between SEQ ID NO: 1 position 438 and 439, sequence GATT between SEQ ID NO: 1 position 438 and 439; the deletion of one or more of: amino acid K at SEQ ID NO: 1 position 252, amino acids QE at SEQ ID NO: 1 positions 435 and 436, and amino acid E at SEQ ID NO: 1 position 445; and/or any combination of replacement, insertion, or deletion provided for herein. In some embodiments, the adenovirus comprises a hexon polypeptide of SEQ ID NO: 2, except that the residue at position 864 is D and/or the residue at position 937 is R as compared to SEQ ID NO: 2. In some embodiments, the adenovirus comprises a hexon polypeptide of SEQ ID NO: 2, except that the residue at position 864 is D and the residue at position 937 is R as compared to SEQ ID NO: 2. In some embodiments, the nucleic acid molecule encodes for the capsid hexon polypeptide of SEQ ID NO: 18. In some embodiments, the nucleic acid molecule comprises a sequence of SEQ ID NO: 17.

(SEQ ID NO: 17)
ATGGCTACCCCTTCGATGATGCCGCAGTGGTCTTACATGCACAT
CTCGGGCCAGGACGCCTCGGAGTACCTGAGCCCCGGGCTGGTGC
AGTTTGCCCGCGCCACCGAGACGTACTTCAGCCTGAATAACAAG
TTTAGAAACCCCACGGTGGCGCCTACGCACGACGTGACCACAGA
CCGGTCCCAGCGTTTGACGCTGCGGTTCATCCCTGTGGACCGTG
AGGATACTGCGTACTCGTACAAGGCGCGGTTCACCCTAGCTGTG
GGTGATAACCGTGTGCTGGACATGGCTTCCACGTACTTTGACAT
CCGCGGCGTGCTGGACAGGGGCCCTACTTTTAAGCCCTACTCTG
GCACTGCCTACAACGCCCTGGCTCCCAAGGGTGCCCCAAATTCA
TGCGAGTGGGATGAAGATGATACTCAGGTACAGGTAGCGGCTGA
AGACGATCAAGACGACGACGAAGAAGAGGAACAACTACCTCAGC
AGAGAAATGGCAAAAAAACTCACGTATATGCTCAGGCACCGTTT
GCTGGCGAAGCAATTAACAAAAACGGCCTGCAGATAGGAACTAA
CGGTGCAGCCACTGAAGGAAATAAGGAAATTTACGCAGATAAAA
CTTATCAACCTGAACCACAAATAGGAGAATCACAGTGGAACGAA
GCCGAATCGTCCGTAGCAGGTGGAAGGGTTCTTAAAAAGACTAC
TCCCATGAAACCATGCTATGGCTCCTATGCCAGACCTACCAATT
CTAACGGAGGTCAGGGCGTTATGGTTGAACAAAATGGTAAATTG
GAAAGTCAAGTAGAAATGCAATTTTTTTCAACTTCTGTAAATGC
TATGAACGAGGCAAACGCTATTCAACCTAAACTAGTGTTGTATA
GTGAAGATGTAAATATGGAAACCCCAGACACTCATCTTTCTTAT
AAGCCTGGAAAAAGTGATGATAATTCTAAGGCAATGTTGGGTCA
ACAATCTATGCCAAACAGACCCAATTACATAGCTTTCAGGGACA
ATTTTATTGGCCTAATGTATTACAACAGCACTGGTAACATGGGT
GTTCTTGCTGGTCAGGCATCACAGCTAAATGCTGTCGTAGATTT
GCAAGACAGAAACACAGAGCTGTCCTACCAACTTTTGCTTGATT
CTATTGGTGATCGAACCAGATACTTTTCCATGTGGAATCAGGCT
GTAGACAGCTACGATCCAGATGTTAGAATTATCGAGAACCATGG
AACTGAGGATGAATTGCCAAATTATTGTTTTCCTCTTGGCGGAA
TTGGGGTGACGGACACCTATCAAGCTATTAAGGCTACAAATGGA
AATGGAGGCGCCACTACCTGGGCTCAGGACAATACTTTTGCAGA
ACGAAATGAAATAGGGGTGGGAAATAACTTTGCCATGGAAATTA
ACCTGAATGCCAACCTATGGAGAAATTTCCTTTACTCCAATATT
GCGCTGTACCTGCCAGACAAGCTAAAATACAACCCCACCAATGT
GGAAATTTCTGATAACCCAAACACCTACGACTACATGAACAAGC
GAGTGGTGGCTCCCGGGCTAGTGGACTGCTACATTAACCTTGGA
GCACGCTGGTCCCTTGACTATATGGACAACGTCAACCCATTTAA
CCACCACCGCAATGCTGGCCTGCGCTACCGCTCAATGTTGCTGG
GCAATGGTCGCTATGTGCCCTTCCACATCCAGGTGCCTCAGAAG
TTCTTTGCCATTAAAAACCTCCTTCTCCTGCCGGGCTCATACAC
CTACGAGTGGAACTTCAGGAAGGATGTTAACATGGTTCTGCAGA
GCTCCCTAGGAAATGACCTAAGGGTTGACGGAGCCAGCATTAAG
TTTGATAGCATTTGCCTTTACGCCACCTTCTTCCCCATGGCCCA
CAACACCGCCTCCACGCTTGAGGCCATGCTTAGAAACGACACCA
ACGACCAGTCCTTTAACGACTATCTCTCCGCCGCCAACATGCTC
TACCCTATACCCGCCAACGCTACCAACGTGCCCATATCCATCCC
CTCCCGCAACTGGGCGGCTTTCCGCGGCTGGGCCTTCACGCGCC
TTAAGACTAAGGAAACCCCATCACTGGGCTCGGGCTACGACCCT
TATTACACCTACTCTGGCTCTATACCCTACCTAGATGGAACCTT
TTACCTCAACCACACCTTTAAGAAGGTGGCCATTACCTTTGACT
CTTCTGTCAGCTGGCCTGGCAATGACCGCCTGCTTACCCCCAAC
GAGTTTGAAATTAAGCGCTCAGTTGACGGGGAGGGTTACAACGT
TGCCCAGTGTAACATGACCAAAGACTGGTTCCTGGTACAAATGC
TAGCTAACTATAACATTGGCTACCAGGGCTTCTATATCCCAGAG
AGCTACAAGGACCGCATGTACTCCTTCTTTAGAAACTTCCAGCC
CATGAGCCGTCAGGTGGTGGATGATACTAAATACAAGGACTACC
AACAGGTGGGCATCCTACACCAACACAACAACTCTGGATTTGTT
GGCTACCTTGCCCCCACCATGCGCGAAGGACAGGCCTACCCTGC
TAACTTCCCCTATCCGCTTATAGGCAAGACCGCAGTTGACAGCA
TTACCCAGAAAAAGTTTCTTTGCGATCGCACCCTTTGGCGCATC
CCATTCTCCAGTAACTTTATGTCCATGGGCGCACTCACAGACCT
GGGCCAAAACCTTCTCTACGCCAACTCCGCCCACGCGCTAGACA
TGACTTTTGAGGTGGATCCCATGGACGAGCCCACCCTTCTTTAT
GTTTTGTTTGAAGTCTTTGACGTGGTCCGTGTGCACCAGCCGCA
CCGCGGCGTCATCGAAACCGTGTACCTGCGCACGCCCTTCTCGG
CCGGCAACGCCACAACATAA

SEQ ID NO: 17 is a non-limiting example of such a nucleic acid molecule and due to the degenerate nature of the codons, other nucleic acid molecules encoding for SEQ ID NO: 18 can also be used.

In some embodiments, any nucleic acid molecule encoding a capsid hexon polypeptide of the Ad strain Ad5 provided for herein does not comprise an insertion, deletion, or substitution at or between residue T at position 166 and residue F at position 169 of SEQ ID NO: 1; residue H at position 218 and residue A at position 220 of SEQ ID NO: 1; residue F at position 267 and residue S at position 268 of SEQ ID NO: 1; residue G at position 434 and residue Q at position 435 of SEQ ID NO: 1; and/or any combination thereof.

In some embodiments, a vector comprises any of the nucleic acid molecules provided for herein. In some embodiments, the vector is an adenoviral vector comprising a transgene encoding, for example, a heterologous polypeptide. In some embodiments, the heterologous polypeptide is any of the polypeptides or combination of polypeptides provided for herein. In some embodiments, the adenoviral vector further comprises at least one of an E1 deletion and an E3 deletion. In some embodiments, a recombinant cell comprising any of the vectors provided for herein is provided. In some embodiments, an immunogenic composition comprising any of the vectors provided for herein is provided.

In some embodiments, a recombinant Ad strain Ad5 hexon polypeptide comprising at least one capsid HVR polypeptide from Ad strain Ad57 is provided. In some embodiments, the recombinant Ad strain Ad5 hexon polypeptide comprises at least one capsid hexon HVR polypeptide from Ad strain Ad57. In some embodiments, the at least one capsid hexon polypeptides of the Ad strain Ad5 comprises one or more capsid hexon HVR polypeptides from Ad strain Ad57 provided for herein. In some embodiments, the capsid hexon polypeptides of the Ad strain Ad5 comprises one or more capsid hexon HVR polypeptides from Ad strain Ad57, or any of the insertions or deletions provided for herein. In some embodiments, SEQ ID NO: 1 or any Ad5 hexon sequence known in the art can be modified by the replacement of one or more amino acid sequences by of one or more of SEQ ID NOs: 4, 8, 10, 12, 14, or 16 as described herein; the insertion of one or more of: sequence EQ between SEQ ID NO: 1 position 156 and 157, SEQ ID NO: 5 between SEQ ID NO: 1 position 187 and 188, sequence TT between SEQ ID NO: 1 position 438 and 439, sequence GATT between SEQ ID NO: 1 position 438 and 439; the deletion of one or more of: amino acid K at SEQ ID NO: 1 position 252, amino acids QE at SEQ ID NO: 1 positions 435 and 436, and amino acid E at SEQ ID NO: 1 position 445; and/or any combination of replacement, insertion, or deletion provided for herein. In some embodiments, the adenovirus comprises a hexon polypeptide of SEQ ID NO: 2, except that the residue at position 864 is D and/or the residue at position 937 is R as compared to SEQ ID NO: 2. In some embodiments, the adenovirus comprises a hexon polypeptide of SEQ ID NO: 2, except that the residue at position 864 is D and the residue at position 937 is R as compared to SEQ ID NO: 2. In some embodiments, recombinant Ad strain Ad5 hexon polypeptide comprises SEQ ID NO: 18.

In some embodiments, any recombinant polypeptide provided for herein does not comprise an insertion, deletion, or substitution at or between residue T at position 166 and residue F at position 169 of SEQ ID NO: 1; residue H at position 218 and residue A at position 220 of SEQ ID NO: 1; residue F at position 267 and residue S at position 268 of SEQ ID NO: 1; residue G at position 434 and residue Q at position 435 of SEQ ID NO: 1; and/or any combination thereof.

In some embodiments, a virus particle comprising any of the polypeptides or recombinant polypeptides provided for herein is provided. In some embodiments, the virus particle is an adenovirus particle. In some embodiments, the virus particle further comprises a polynucleotide encoding a transgene or a heterologous protein.

In some embodiments, a pharmaceutical composition comprising any of the polypeptides or recombinant Ads provided for herein is provided. In some embodiments, a pharmaceutical composition comprises any recombinant Ads or polypeptides provided for herein and a pharmaceutically acceptable carrier. Pharmaceutically acceptable carries are known in the art, and can comprises suspension agents, buffering agents, or other pharmaceutically acceptable emulsions, solutions, suspensions, syrups, and diluents. In some embodiments, the pharmaceutically acceptable carrier is water.

In some embodiments, any recombinant Ad provided for herein has an increase in transduction in the spleen as compared to the liver. In some embodiments, recombinant Ad provided for herein as an increased in transduction in the spleen as compared to the liver, as compared to a recombinant Ad comprising the hexon polypeptide having the amino acid sequence of SEQ ID NO: 1.

In some embodiments, any recombinant Ad provided for herein has an increased liver safety profile. In some embodiments, the increased liver safety profile is in comparison to a recombinant Ad comprising the hexon polypeptide having the amino acid sequence of SEQ ID NO: 1. In some embodiments, any recombinant Ad provided for herein does not significantly elevate liver enzymes. In some embodiments, the liver enzymes are ALT and AST.

### Methods

In some embodiments, a method of producing a vector is provided, the method comprising; (a) growing a recombinant cell under conditions for production of the vector; and (b) isolating the vector from the recombinant cell. In some embodiments, the recombinant cell is a cell line, a mixed cell line, an immortalized cell or clonal population of immortalized cells, as well known in the art. The recombinant cell chosen for expression may be of mammalian origin or may be selected from COS-1, COS-7, HEK293, 911, BHK21, CHO, BSC-1, He G2, SP2/0, HeLa, LP293, AE1-2a, N52.E6, PERC.6, A549, high capacity Ad vector producer cell lines using recombinase systems, E2T, C7, myeloma, lymphoma, yeast, insect or plant cells, or any derivative, immortalized or transformed cell thereof. In some embodiments, the recombinant cell has specific modifications beneficial to the production of the vector.

In some embodiments, a method of inducing an immune response in a subject in need thereof is provided, comprising administering to the subject an immunogenic composition. In some embodiments, the immunogenic composition is any immunogenic composition provided for herein, such as a recombinant Ad comprising a hexon polypeptide as provided for herein

In some embodiments, a method of treating or preventing a viral infection is provided, the method comprising administering a recombinant Ad, wherein the recombinant Ad expresses a heterologous viral protein. In some embodiments, the recombinant Ad is any recombinant Ad provided for herein. In some embodiments, the heterologous viral protein expressed by the recombinant Ad is any recombinant Ad provided for herein. In some embodiments, the heterologous viral protein is from HBV, HPV, EBV, CMV, HTLV, or Polyoma viral protein. In some embodiments, the recombinant Ad has an increase in transduction in the spleen as compared to the liver. In some embodiments, the recombinant Ad has an increase in transduction in the spleen as compared to the liver, as compared to a recombinant Ad comprising the hexon polypeptide having the amino acid sequence of SEQ ID NO: 1. In some embodiments, the recombinant Ad comprising the hexon polypeptide does not increase liver enzymes when administered to a subject as compared to a recombinant Ad comprising the hexon polypeptide having the amino acid sequence of SEQ ID NO: 1.

In some embodiments, a method of treating a cancer is provided, the method comprising administering any recombinant adenovirus provided for herein. In some embodiments, the method comprises administering any recombinant adenovirus provided for herein, wherein the recombinant Ad expresses a cytokine, an immunomodulatory protein, and/or a tumor antigen. In some embodiments, the recombinant Ad has an increase in transduction in the spleen as compared to the liver. In some embodiments, the recombinant Ad has an increase in transduction in the spleen as compared to the liver, as compared to a recombinant Ad comprising the hexon polypeptide having the amino acid sequence of SEQ ID NO: 1. In some embodiments, the recombinant Ad comprising the hexon polypeptide does not increase liver enzymes when administered to a subject as compared to a recombinant Ad comprising the hexon polypeptide having the amino acid sequence of SEQ ID NO: 1.

### Enumerated Embodiments

Embodiments provided herein also include, but are not limited to, the following:
1. A recombinant adenovirus (Ad), wherein capsid hexon polypeptides of an Ad strain Ad5 comprise at least one capsid hexon hypervariable region (HVR) polypeptide from Ad strain Ad57.
2. The recombinant Ad of embodiment 1, wherein the capsid hexon polypeptides of the Ad strain Ad5 comprises a variant of a polypeptide comprising the amino acid sequence of SEQ ID NO: 1, wherein the variant comprises:
   i) an amino acid sequence of SEQ ID NO: 4, SEQ ID NO: 8, SEQ ID NO: 10, SEQ ID NO: 12, SEQ ID NO: 14, or SEQ ID NO: 16, or any combination thereof;
   ii) one or more insertions of:
      a polypeptide of EQ (Glu-Gln) between positions 156 and 157 of SEQ ID NO:1,
      a polypeptide comprising amino acid sequence SEQ ID NO: 5 between positions 187 and 188 of SEQ ID NO: 1,
      a polypeptide of TT (Thr-Thr) between positions 438 and 439 of SEQ ID NO: 1, or
      a polypeptide of GATT (Gly-Ala-Thr-Thr; SEQ ID NO: 6) between positions 438 and 439 of SEQ ID NO: 1;
   iii) one or more deletions of:
      K252 of SEQ ID NO: 1,
      polypeptide Q435E436 of SEQ ID NO: 1, or
      E445 of SEQ ID NO: 1; or
   any combination of replacements, insertions, and deletions of the foregoing.
3. The recombinant Ad of embodiments 1 or 2, wherein the hexon polypeptide of Ad strain Ad5 comprising the amino acid sequence of AATALEINLE (SEQ ID NO: 3) has SEQ ID NO: 3 replaced by an amino acid sequence of DDTQVQVAAE (SEQ ID NO: 4).
4. The recombinant Ad of any one of embodiments 1-3, wherein the hexon polypeptide of Ad strain Ad5 comprises the polypeptide of EQ between residue E (Glu) at position 156 and residue V (Val) at position 157 of SEQ ID NO: 1.
5. The recombinant Ad of any one of embodiments 1-4, wherein the hexon polypeptide of Ad strain Ad5 does not comprise an insertion, deletion, or substitution at, or between, residue T (Thr) at position 166 and residue F (Phe) at position 169 of SEQ ID NO: 1.
6. The recombinant Ad of any one of embodiments 1-5, wherein the hexon polypeptide of Ad strain Ad5 comprises an insertion of a polypeptide comprising the amino acid sequence of TNGAA (SEQ ID NO: 5) between residue G (Gly) at position 187 and residue V (Val) at position 188 of SEQ ID NO: 1.
7. The recombinant Ad of any one of embodiments 1-6, wherein the hexon polypeptide of Ad strain Ad5 does not comprise an insertion, deletion, or substitution at or between residue H (His) at position 218 and residue A (Ala) at position 220 of SEQ ID NO: 1.
8. The recombinant Ad of any one of embodiments 1-7, wherein the hexon polypeptide of Ad strain Ad5 comprises a deletion of residue K (Lys) at position 252 of SEQ ID NO: 1.
9. The recombinant Ad of any one of embodiments 1-8, wherein the hexon polypeptide of Ad strain Ad5 does not comprise an insertion, deletion, or substitution at or between residue F (Phe) at position 267 and residue S (Ser) at position 268 of SEQ ID NO: 1.
10. The recombinant Ad of any one of embodiments 1-9, wherein the hexon polypeptide of Ad strain Ad5 does not comprise an insertion, deletion, or substitution at or between residue G (Gly) at position 434 and residue Q (Gln) at position 435 of SEQ ID NO: 1.
11. The recombinant Ad of any one of embodiments 1-10, wherein the hexon polypeptide of Ad strain Ad5 comprises a deletion of residues QE at positions 435 and 436 of SEQ ID NO: 1.
12. The recombinant Ad of any one of embodiments 1-11, wherein the hexon polypeptide of Ad strain Ad5 comprises an insertion of a polypeptide comprising the sequence of TT between residue G at position 438 and residue W (Trp) at position 439 of SEQ ID NO: 1.
13. The recombinant Ad of any one of embodiments 1-12, wherein the hexon polypeptide of Ad strain Ad5 comprises an insertion of a polypeptide comprising the amino acid sequence of GATT (SEQ ID NO: 6) between residue G at position 438 and residue W at position 439 of SEQ ID NO: 1.
14 The recombinant Ad of any one of embodiments 1-12, wherein the hexon polypeptide of Ad strain Ad5 comprises a deletion of residue E at position 445 of SEQ ID NO: 1.
15. The recombinant Ad of any one of embodiments 1-14, wherein the hexon polypeptide of Ad strain Ad5 comprising the amino acid sequence of SEQ ID NO: 7 has the amino acid sequence of SEQ ID NO: 7 replaced by the amino acid sequence of SEQ ID NO: 8.
16. The recombinant Ad of any one of embodiments 1-15, wherein the hexon polypeptide of Ad strain Ad5 comprising the amino acid sequence of SEQ ID NO: 9 has the amino acid sequence of SEQ ID NO: 9 replaced by the amino acid sequence of SEQ ID NO: 10.
17. The recombinant Ad of any one of embodiments 1-16, wherein the hexon polypeptide of Ad strain Ad5 comprising the amino acid sequence of SEQ ID NO: 11 has the amino acid sequence of SEQ ID NO: 11 replaced by the amino acid sequence of SEQ ID NO: 12.
18. The recombinant Ad of any one of embodiments 1-17, wherein the hexon polypeptide of Ad strain Ad5 comprising the amino acid sequence of SEQ ID NO: 13 has the amino acid sequence of SEQ ID NO: 13 replaced by the amino acid sequence of SEQ ID NO: 14.
19. The recombinant Ad of any one of embodiments 1-18, wherein the hexon polypeptide of Ad strain Ad5 comprising the amino acid sequence of SEQ ID NO: 15 has the amino acid sequence of SEQ ID NO: 15 replaced by the amino acid sequence of SEQ ID NO: 16.
20. The recombinant Ad of any one of embodiments 1-19, wherein the hexon polypeptide of Ad strain Ad5 comprises a variant of the amino acid sequence of SEQ ID NO: 2 or SEQ ID NO: 18, wherein the variant comprises an aspartic acid (D) residue at position 864 and/or an arginine (R) residue at position 937 as compared to SEQ ID NO: 2 or SEQ ID NO: 18.
21. The recombinant Ad of embodiment 1, wherein the hexon polypeptide comprises an amino acid sequence of SEQ ID NO: 18.
22. The recombinant Ad of any one of embodiments 1-20, wherein the hexon polypeptide comprises a variant of a polypeptide comprising the amino acid sequence of SEQ ID NO: 18.
23. The recombinant Ad of embodiment 22, wherein variant hexon polypeptide comprises an amino acid sequence that is at least 75, 80, 85, 90, 91, 92, 93, 94, 95, 96, 97, 98, or 99% identical to the amino acid sequence of SEQ ID NO: 18.
24. The recombinant Ad of embodiment 22, wherein the variant hexon polypeptide comprises an amino acid sequence that is at least 75%, 80%, 85%, 90%, 91%, 92%, 93%, 94%, 95%, 96%, 97%, 98%, or 99% identical to the amino acid sequence of SEQ ID NO: 18, provided that the variant comprises:
   the amino acid sequence of SEQ ID NO: 4, SEQ ID NO: 8, SEQ ID NO: 10, SEQ ID NO: 12, SEQ ID NO: 14, and SEQ ID NO: 16; or
   the variant comprises an aspartic acid (D) residue at position 864 and/or an arginine (R) residue at position 937.
25. The recombinant Ad of embodiment 22, wherein the variant hexon polypeptide comprises 1, 2, 3, 4, 5, 6, 7, 8, 9, or 10 mutations (e.g., substitutions, insertions, or deletions), as compared to the amino acid sequence of SEQ ID NO: 18, provided that the variant comprises:
   the amino acid sequence of SEQ ID NO: 4, SEQ ID NO: 8, SEQ ID NO: 10, SEQ ID NO: 12, SEQ ID NO: 14, and SEQ ID NO: 16; or
   the variant comprises an aspartic acid (D) residue at position 864 and/or an arginine (R) residue at position 937.
26. The recombinant Ad of any one of embodiments 22-25, wherein the variant hexon polypeptide has increased in transduction in the spleen as compared to the liver.
27. The recombinant Ad of any one of embodiments 22-25, wherein the variant hexon polypeptide has increased in transduction in the spleen as compared to the liver as compared to a recombinant Ad comprising the hexon polypeptide having the amino acid sequence of SEQ ID NO: 1.
28. The recombinant Ad of any one of embodiments 22-25, wherein the variant hexon polypeptide does not increase liver enzymes when administered to a subject as compared to a recombinant Ad comprising the hexon polypeptide having the amino acid sequence of SEQ ID NO: 1
29. A recombinant Ad of any one of embodiments 1-28 further comprising a polynucleotide encoding a heterologous protein.
30. The recombinant Ad of embodiment 29, wherein the heterologous protein is a cytokine, an immunoglobulin, an immunomodulatory protein, a viral protein, a patient specific neoepitope, or any combination thereof.
31. The recombinant Ad of embodiment 30, wherein the viral protein is from HBV, HPV, EBV, CMV, HTLV, Polyoma, or any combination thereof.
32. The recombinant Ad of embodiments 30 or 31, wherein the immunomodulatory protein is from the B7 family.
33. The recombinant Ad of embodiment 32, wherein the immunomodulatory protein is B7.1 or B7.2.
34. The recombinant Ad of embodiments 32 and 33, wherein the immunomodulatory protein from the B7 family is fused to a domain from an immunoglobulin protein.
35. The recombinant Ad of embodiments 30-34, wherein the patient specific neoepitope is a tumor-specific antigen derived from somatic tumor mutation from a patient.
36. The recombinant Ad of any one of embodiments 1-35, further comprising a targeting moiety.
37. The recombinant Ad of embodiment 36, wherein the targeting moiety is a moiety that targets the Ad to carbohydrates, cell membranes and cells selected from muscle cells, tumors, cancer cells, kidney cells, liver cells, or mucosal cells.
38. A recombinant Ad of any one of embodiments 1-37, wherein the adenovirus is replication competent or is a conditionally-replicating Adenovirus (CRAd) that comprises a modified E1A gene encoding an E1A polypeptide, wherein the CRAd exhibits amino acid substitutions in the E1A polypeptide relative to wild-type E1A polypeptide of an Ad strain.
39. A nucleic acid molecule encoding capsid hexon polypeptides of an Ad strain Ad5 comprising at least one capsid hexon HVR polypeptide from Ad strain Ad57.
40. The nucleic acid molecule of embodiment 39, wherein the capsid hexon polypeptides of the Ad strain Ad5 comprises a variant of a polypeptide comprising the amino acid sequence of SEQ ID NO: 1, wherein the variant comprises:
   i) an amino acid sequence of SEQ ID NO: 4, SEQ ID NO: 8, SEQ ID NO: 10, SEQ ID NO: 12, SEQ ID NO: 14, or SEQ ID NO: 16, or any combination thereof;
   ii) one or more insertions of:
      a polypeptide of EQ (Glu-Gln) between positions 156 and 157 of SEQ ID NO:1,
      a polypeptide comprising amino acid sequence SEQ ID NO: 5 between positions 187 and 188 of SEQ ID NO: 1,
      a polypeptide of TT (Thr-Thr) between positions 438 and 439 of SEQ ID NO: 1, or
      a polypeptide of GATT (Gly-Ala-Thr-Thr; SEQ ID NO: 6) between positions 438 and 439 of SEQ ID NO: 1;
   iii) one or more deletions of:
      K252 of SEQ ID NO: 1,
      polypeptide Q435E436 of SEQ ID NO: 1, or
      E445 of SEQ ID NO: 1; or
   any combination of replacements, insertions, and deletions of the foregoing.
41. The nucleic acid molecule of embodiments 39 or 40, wherein the hexon polypeptide of Ad strain Ad5 comprising the amino acid sequence of AATALEINLE (SEQ ID NO: 3) has SEQ ID NO: 3 replaced by an amino acid sequence of DDTQVQVAAE (SEQ ID NO: 4).
42. The nucleic acid molecule of embodiments 39-41, wherein the hexon polypeptide of Ad strain Ad5 comprises the polypeptide of EQ between residue E (Glu) at position 156 and residue V (Val) at position 157 of SEQ ID NO: 1.
43. The nucleic acid molecule of any one of embodiments 39-42, wherein the hexon polypeptide of Ad strain Ad5 does not comprise an insertion, deletion, or substitution at, or between, residue T (Thr) at position 166 and residue F (Phe) at position 169 of SEQ ID NO: 1.
44. The nucleic acid molecule of any one of embodiments 39-44, wherein the hexon polypeptide of Ad strain Ad5 comprises an insertion of a polypeptide comprising the amino acid sequence of TNGAA (SEQ ID NO: 5) between residue G (Gly) at position 187 and residue V (Val) at position 188 of SEQ ID NO: 1.
45. The nucleic acid molecule of any one of embodiments 39-44, wherein the hexon polypeptide of Ad strain Ad5 does not comprise an insertion, deletion, or substitution at or between residue H (His) at position 218 and residue A (Ala) at position 220 of SEQ ID NO: 1.
46. The nucleic acid molecule of any one of embodiments 39-45, wherein the hexon polypeptide of Ad strain Ad5 comprises a deletion of residue K (Lys) at position 252 of SEQ ID NO: 1.
47. The nucleic acid molecule of any one of embodiments 39-46, wherein the hexon polypeptide of Ad strain Ad5 does not comprise an insertion, deletion, or substitution at or between residue F (Phe) at position 267 and residue S (Ser) at position 268 of SEQ ID NO: 1.
48. The nucleic acid molecule of any one of embodiments 39-47, wherein the hexon polypeptide of Ad strain Ad5 does not comprise an insertion, deletion, or substitution at or between residue G (Gly) at position 434 and residue Q (Gln) at position 435 of SEQ ID NO: 1.
49. The nucleic acid molecule of any one of embodiments 39-48, wherein the hexon polypeptide of Ad strain Ad5 comprises a deletion of residues QE at positions 435 and 436 of SEQ ID NO: 1.
50. The nucleic acid molecule of any one of embodiments 39-49, wherein the hexon polypeptide of Ad strain Ad5 comprises an insertion of a polypeptide comprising the sequence of TT between residue G at position 438 and residue W (Trp) at position 439 of SEQ ID NO: 1.
51. The nucleic acid molecule of any one of embodiments 39-50, wherein the hexon polypeptide of Ad strain Ad5 comprises an insertion of a polypeptide comprising the amino acid sequence of GATT (SEQ ID NO: 6) between residue G at position 438 and residue W at position 439 of SEQ ID NO: 1.
52. The nucleic acid molecule of any one of embodiments 39-51, wherein the hexon polypeptide of Ad strain Ad5 comprises a deletion of residue E at position 445 of SEQ ID NO: 1.
53. The nucleic acid molecule of any one of embodiments 39-52, wherein the hexon polypeptide of Ad strain Ad5 comprising the amino acid sequence of SEQ ID NO: 7 has the amino acid sequence of SEQ ID NO: 7 replaced by the amino acid sequence of SEQ ID NO: 8.
54. The nucleic acid molecule of any one of embodiments 39-53, wherein the hexon polypeptide of Ad strain Ad5 comprising the amino acid sequence of SEQ ID NO: 9 has the amino acid sequence of SEQ ID NO: 9 replaced by the amino acid sequence of SEQ ID NO: 10.
55. The nucleic acid molecule of any one of embodiments 39-54, wherein the hexon polypeptide of Ad strain Ad5 comprising the amino acid sequence of SEQ ID NO: 11 has the amino acid sequence of SEQ ID NO: 11 replaced by the amino acid sequence of SEQ ID NO: 12.
56. The nucleic acid molecule of any one of embodiments 39-55, wherein the hexon polypeptide of Ad strain Ad5 comprising the amino acid sequence of SEQ ID NO: 13 has the amino acid sequence of SEQ ID NO: 13 replaced by the amino acid sequence of SEQ ID NO: 14.
57. The nucleic acid molecule of any one of embodiments 39-56, wherein the hexon polypeptide of Ad strain Ad5 comprising the amino acid sequence of SEQ ID NO: 15 has the amino acid sequence of SEQ ID NO: 15 replaced by the amino acid sequence of SEQ ID NO: 16.
58. The nucleic acid molecule of any one of any one of embodiments 39-57, wherein the hexon polypeptide of Ad strain Ad5 comprises a variant of the amino acid sequence of SEQ ID NO: 2 or SEQ ID NO: 18, wherein the variant comprises an aspartic acid (D) residue at position 864 and/or an arginine (R) residue at position 937 as compared to SEQ ID NO: 2 or SEQ ID NO: 18.
59. The nucleic acid molecule of any one of embodiments 39-58, wherein the nucleic acid molecule comprises a sequence of SEQ ID NO: 17.
60. The nucleic acid molecule of any one of embodiments 39-59, wherein the nucleic acid molecule encodes for a variant of a hexon polypeptide comprising the amino acid sequence of SEQ ID NO: 18.
61. The nucleic acid molecule of embodiment 60, wherein variant hexon polypeptide comprises an amino acid sequence that is at least 75, 80, 85, 90, 91, 92, 93, 94, 95, 96, 97, 98, or 99% identical to the amino acid sequence of SEQ ID NO: 18.
62. The nucleic acid molecule of embodiment 60, wherein the variant hexon polypeptide comprises an amino acid sequence that is at least 75%, 80%, 85%, 90%, 91%, 92%, 93%, 94%, 95%, 96%, 97%, 98%, or 99% identical to the amino acid sequence of SEQ ID NO: 18, provided that the variant comprises:
   the amino acid sequence of SEQ ID NO: 4, SEQ ID NO: 8, SEQ ID NO: 10, SEQ ID NO: 12, SEQ ID NO: 14, and SEQ ID NO: 16; or
   the variant comprises an aspartic acid (D) residue at position 864 and/or an arginine (R) residue at position 937.
63. The nucleic acid molecule of embodiment 60, wherein the variant hexon polypeptide comprises 1, 2, 3, 4, 5, 6, 7, 8, 9, or 10 mutations (e.g., substitutions, insertions, or deletions), as compared to the amino acid sequence of SEQ ID NO: 18, provided that the variant comprises:
   the amino acid sequence of SEQ ID NO: 4, SEQ ID NO: 8, SEQ ID NO: 10, SEQ ID NO: 12, SEQ ID NO: 14, and SEQ ID NO: 16; or
   the variant comprises an aspartic acid (D) residue at position 864 and/or an arginine (R) residue at position 937.
64. The nucleic acid molecule of any one of embodiments 60-63, wherein the variant hexon polypeptide has increased in transduction in the spleen as compared to the liver.
65. The nucleic acid molecule of any one of embodiments 60-64, wherein the variant hexon polypeptide has increased in transduction in the spleen as compared to the liver as compared to a recombinant Ad comprising the hexon polypeptide having the amino acid sequence of SEQ ID NO: 1.
66. The nucleic acid molecule of any one of embodiments 60-64, wherein the variant hexon polypeptide does not increase liver enzymes when administered to a subject as compared to a recombinant Ad comprising the hexon polypeptide having the amino acid sequence of SEQ ID NO: 1
67. A vector comprising the nucleic acid molecule of any one of any one of embodiments 39-66.
68. The vector of embodiment 67, wherein the vector is an Ad vector comprising a transgene encoding, for example, a heterologous polypeptide.
69. The vector of embodiment 68, wherein the Ad vector further comprises at least one of an E1 deletion and an E3 deletion.
70. A recombinant cell comprising the vector of any one of embodiments 67-69.
71. A method of producing a vector, comprising; (a) growing the recombinant cell of embodiment 70 under conditions for production of the vector; and (b) isolating the vector from the recombinant cell.
72. An immunogenic composition comprising the vector of any one of embodiments 67-69.
73. A method of inducing an immune response in a subject in need thereof, comprising administering to the subject the immunogenic composition of embodiment 72.
74. The method of embodiment 73, wherein the immune response is induced without significantly increasing liver enzymes in the subject.
75. The method of embodiment 74, wherein the immune response is induced without significantly increasing liver enzymes in the subject as compared to a vector comprising a hexon polypeptide having the amino acid sequence of SEQ ID NO: 1.
76. The method of any one of embodiments 73-75, wherein the composition is administered subcutaneously or intravenously.
77. A recombinant Ad strain Ad5 hexon polypeptide comprising at least one capsid hexon hypervariable region (HVR) polypeptide from Ad strain Ad57.
78. The recombinant hexon polypeptide of embodiment 77, wherein the capsid hexon polypeptides of the Ad strain Ad5 comprises a variant of a polypeptide comprising the amino acid sequence of SEQ ID NO: 1, wherein the variant comprises:
   i) an amino acid sequence of SEQ ID NO: 4, SEQ ID NO: 8, SEQ ID NO: 10, SEQ ID NO: 12, SEQ ID NO: 14, or SEQ ID NO: 16, or any combination thereof;
   ii) one or more insertions of:
      a polypeptide of EQ (Glu-Gln) between positions 156 and 157 of SEQ ID NO:1,
      a polypeptide comprising amino acid sequence SEQ ID NO: 5 between positions 187 and 188 of SEQ ID NO: 1,
      a polypeptide of TT (Thr-Thr) between positions 438 and 439 of SEQ ID NO: 1, or
      a polypeptide of GATT (Gly-Ala-Thr-Thr; SEQ ID NO: 6) between positions 438 and 439 of SEQ ID NO: 1;
   iii) one or more deletions of:
      K252 of SEQ ID NO: 1,
      polypeptide Q435E436 of SEQ ID NO: 1, or
      E445 of SEQ ID NO: 1; or
   any combination of replacements, insertions, and deletions of the foregoing.
79. The recombinant hexon polypeptide of embodiments 77 or 78, wherein the hexon polypeptide of Ad strain Ad5 comprising the amino acid sequence of AATALEINLE (SEQ ID NO: 3) has SEQ ID NO: 3 replaced by an amino acid sequence of DDTQVQVAAE (SEQ ID NO: 4).
80. The recombinant hexon polypeptide of any one of embodiments 77-79, wherein the hexon polypeptide of Ad strain Ad5 comprises the polypeptide of EQ between residue E (Glu) at position 156 and residue V (Val) at position 157 of SEQ ID NO: 1.
81. The recombinant hexon polypeptide of any one of embodiments 77-80, wherein the hexon polypeptide of Ad strain Ad5 does not comprise an insertion, deletion, or substitution at, or between, residue T (Thr) at position 166 and residue F (Phe) at position 169 of SEQ ID NO: 1.
82. The recombinant hexon polypeptide of any one of embodiments 77-81, wherein the hexon polypeptide of Ad strain Ad5 comprises an insertion of a polypeptide comprising the amino acid sequence of TNGAA (SEQ ID NO: 5) between residue G (Gly) at position 187 and residue V (Val) at position 188 of SEQ ID NO: 1.
83. The recombinant hexon polypeptide of any one of embodiments 77-82, wherein the hexon polypeptide of Ad strain Ad5 does not comprise an insertion, deletion, or substitution at or between residue H (His) at position 218 and residue A (Ala) at position 220 of SEQ ID NO: 1.
84. The recombinant hexon polypeptide of any one of embodiments 77-83, wherein the hexon polypeptide of Ad strain Ad5 comprises a deletion of residue K (Lys) at position 252 of SEQ ID NO: 1.
85. The recombinant hexon polypeptide of any one of embodiments 77-84, wherein the hexon polypeptide of Ad strain Ad5 does not comprise an insertion, deletion, or substitution at or between residue F (Phe) at position 267 and residue S (Ser) at position 268 of SEQ ID NO: 1.
86. The recombinant hexon polypeptide of any one of embodiments 77-85, wherein the hexon polypeptide of Ad strain Ad5 does not comprise an insertion, deletion, or substitution at or between residue G (Gly) at position 434 and residue Q (Gln) at position 435 of SEQ ID NO: 1.
87. The recombinant hexon polypeptide of any one of embodiments 77-86, wherein the hexon polypeptide of Ad strain Ad5 comprises a deletion of residues QE at positions 435 and 436 of SEQ ID NO: 1.
88. The recombinant hexon polypeptide of any one of embodiments 77-87, wherein the hexon polypeptide of Ad strain Ad5 comprises an insertion of a polypeptide comprising the sequence of TT between residue G at position 438 and residue W (Trp) at position 439 of SEQ ID NO: 1.
89. The recombinant hexon polypeptide of any one of embodiments 77-88, wherein the hexon polypeptide of Ad strain Ad5 comprises an insertion of a polypeptide comprising the amino acid sequence of GATT (SEQ ID NO: 6) between residue G at position 438 and residue W at position 439 of SEQ ID NO: 1.
90. The recombinant hexon polypeptide of any one of embodiments 77-89, wherein the hexon polypeptide of Ad strain Ad5 comprises a deletion of residue E at position 445 of SEQ ID NO: 1.
91. The recombinant hexon polypeptide of any one of embodiments 77-90, wherein the hexon polypeptide of Ad strain Ad5 comprising the amino acid sequence of SEQ ID NO: 7 has the amino acid sequence of SEQ ID NO: 7 replaced by the amino acid sequence of SEQ ID NO: 8.
92. The recombinant hexon polypeptide of any one of embodiments 77-91, wherein the hexon polypeptide of Ad strain Ad5 comprising the amino acid sequence of SEQ ID NO: 9 has the amino acid sequence of SEQ ID NO: 9 replaced by the amino acid sequence of SEQ ID NO: 10.
93. The recombinant hexon polypeptide of any one of embodiments 77-92, wherein the hexon polypeptide of Ad strain Ad5 comprising the amino acid sequence of SEQ ID NO: 11 has the amino acid sequence of SEQ ID NO: 11 replaced by the amino acid sequence of SEQ ID NO: 12.
94. The recombinant hexon polypeptide of any one of embodiments 77-93, wherein the hexon polypeptide of Ad strain Ad5 comprising the amino acid sequence of SEQ ID NO: 13 has the amino acid sequence of SEQ ID NO: 13 replaced by the amino acid sequence of SEQ ID NO: 14.
95. The recombinant hexon polypeptide of any one of embodiments 77-94, wherein the hexon polypeptide of Ad strain Ad5 comprising the amino acid sequence of SEQ ID NO: 15 has the amino acid sequence of SEQ ID NO: 15 replaced by the amino acid sequence of SEQ ID NO: 16.
96. The recombinant hexon polypeptide of any one of embodiments 77-95, wherein the hexon polypeptide of Ad strain Ad5 comprises a variant of the amino acid sequence of SEQ ID NO: 2 or SEQ ID NO: 18, wherein the variant comprises an aspartic acid (D) residue at position 864 and/or an arginine (R) residue at position 937 as compared to SEQ ID NO: 2 or SEQ ID NO: 18.
97. The recombinant hexon polypeptide of embodiment 77, wherein the hexon polypeptide comprises an amino acid sequence of SEQ ID NO: 18.
98. The recombinant hexon polypeptide of any one of embodiments 77-96, wherein the hexon polypeptide comprises a variant of a polypeptide comprising the amino acid sequence of SEQ ID NO: 18.
99. The recombinant hexon polypeptide of embodiment 98, wherein variant hexon polypeptide comprises an amino acid sequence that is at least 75, 80, 85, 90, 91, 92, 93, 94, 95, 96, 97, 98, or 99% identical to the amino acid sequence of SEQ ID NO: 18.
100. The recombinant hexon polypeptide of embodiment 98, wherein the variant hexon polypeptide comprises an amino acid sequence that is at least 75%, 80%, 85%, 90%, 91%, 92%, 93%, 94%, 95%, 96%, 97%, 98%, or 99% identical to the amino acid sequence of SEQ ID NO: 18, provided that the variant comprises:
   the amino acid sequence of SEQ ID NO: 4, SEQ ID NO: 8, SEQ ID NO: 10, SEQ ID NO: 12, SEQ ID NO: 14, and SEQ ID NO: 16; or
   the variant comprises an aspartic acid (D) residue at position 864 and/or an arginine (R) residue at position 937.
101. The recombinant hexon polypeptide of embodiment 98, wherein the variant hexon polypeptide comprises 1, 2, 3, 4, 5, 6, 7, 8, 9, or 10 mutations (e.g., substitutions, insertions, or deletions), as compared to the amino acid sequence of SEQ ID NO: 18, provided that the variant comprises:
   the amino acid sequence of SEQ ID NO: 4, SEQ ID NO: 8, SEQ ID NO: 10, SEQ ID NO: 12, SEQ ID NO: 14, and SEQ ID NO: 16; or
   the variant comprises an aspartic acid (D) residue at position 864 and/or an arginine (R) residue at position 937.
102. The recombinant hexon polypeptide of any one of embodiments 98-101, wherein the variant hexon polypeptide has increased in transduction in the spleen as compared to the liver.
103. The recombinant hexon polypeptide of any one of embodiments 98-102, wherein the variant hexon polypeptide has increased in transduction in the spleen as compared to the liver as compared to a recombinant Ad comprising the hexon polypeptide having the amino acid sequence of SEQ ID NO: 1.
104. The recombinant hexon polypeptide of any one of embodiments 98-103, wherein the variant hexon polypeptide does not increase liver enzymes when administered to a subject as compared to a recombinant Ad comprising the hexon polypeptide having the amino acid sequence of SEQ ID NO: 1
105. A virus particle comprising the hexon polypeptide of any one of embodiments 77-104.
106. The virus particle of embodiment 105, wherein the virus particle is an Ad particle.
107. The virus particle of embodiments 105 or 106, further comprising a polynucleotide encoding a transgene or a heterologous protein.
108. A pharmaceutical composition comprising the hexon polypeptide of any one of embodiments 77-104 or the particles of any one of embodiments 105-107.
109. A method of treating a viral infection, the method comprising administering the recombinant hexon polypeptide of any one of embodiments 77-104, the virus particle of embodiments 105-107, or the pharmaceutical composition of embodiment 108.
110. The method of embodiment 109, wherein, when a virus particle is administered, the virus particle expresses a heterologous viral protein.
111. The method of embodiment 110, wherein the heterologous viral protein is from HBV, HPV, EBV, CMV, HTLV, or Polyoma viral protein.
112. The method of embodiments 110 or 111, wherein the virus particle has increased transduction in the spleen as compared to the liver.
113. The method of embodiments 110-112, wherein the virus particle has increased transduction in the spleen as compared to the liver, as compared to a recombinant Ad comprising the hexon polypeptide having the amino acid sequence of SEQ ID NO: 1.
113. A method of treating a cancer, the method comprising administering the recombinant hexon polypeptide of any one of embodiments 77-104, the virus particle of embodiments 105-107, or the pharmaceutical composition of embodiment 108.
114. The method of embodiment 113, wherein, when a virus particle is administered, the virus particle expresses a cytokine, an immunomodulatory protein, and/or a tumor antigen.
115. The method of embodiment 114, wherein the virus particle has increased transduction in the spleen as compared to the liver.
116. The method of embodiments 114 or 115, wherein the virus particle has increased transduction in the spleen as compared to the liver, as compared to a recombinant Ad comprising the hexon polypeptide having the amino acid sequence of SEQ ID NO: 1.

### EXAMPLES

The following examples are illustrative, but not limiting, of the compounds, compositions and methods described herein. Other suitable modifications and adaptations known to those skilled in the art are within the scope of the following embodiments.

### Example 1: Production of a Recombinant Ad Comprising an Ad5/Ad57 Chimeric Hexon Polypeptide

A nucleic acid molecule encoding for a hexon polypeptide comprising SEQ ID NO: 18 is transduced into a HEK293 cells to produce an adenovirus comprising the chimeric hexon polypeptide. The recombinant Ad is also produced comprising a transgene encoding for a HBV polytope.

### Example 2: Recombinant Ad Elicits Robust Antigen-Specific T Cell Reactivity and Enrichment

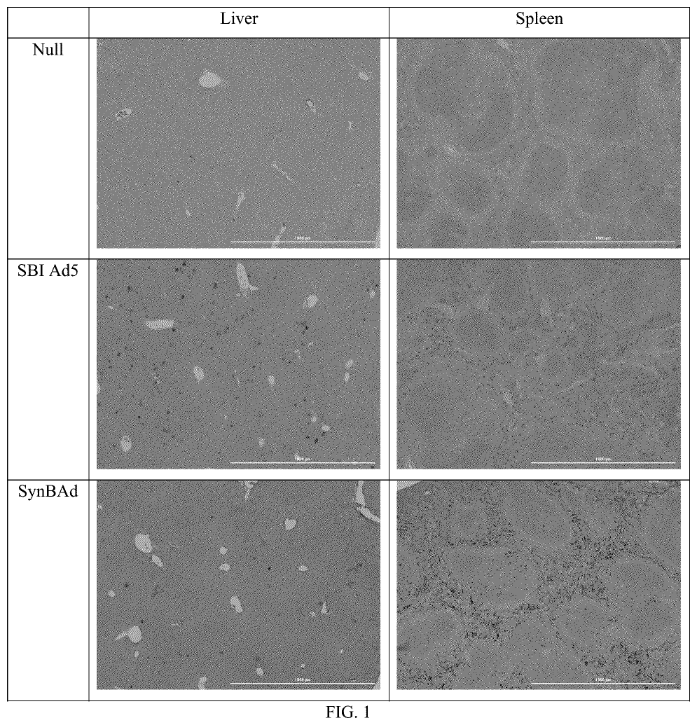

To examine the efficacy of a recombinant Ad5 encoding for a hexon polypeptide comprising SEQ ID NO: 18 (“SynBAd”) was compared to an Ad5 virus comprising a wild-type hexon polypeptide (“SBI Ad5”). First, to test target specificity, both viruses were loaded with a construct encoding luciferase as a reporter and administered intravenously to different groups of C57BL/6 mice at day 0, followed by a second administration at day 21. The animals were acclimated a minimum of 3 days prior to the start of the study, and were housed in microisolators in a 12:12 light/dark cycle. Animals were maintained with a standard rodent chow diet with water and food provided ad libitum. Organs harvested at day 28 showed that both viruses specifically targeted the liver, and did not target the brain, heart, lung, pancreases, kidney, stomach, or colon to any detectable degree. Additionally, histopathology confirms that the liver and spleen were successfully targeted by both viruses, with the SBI Ad5 showing higher transduction and expression of the luciferase in the liver and the SynBAd showing higher transductions and expression of the luciferase in the spleen (see FIG. 1).

### Example 3: SynBAd Elicits Robust Splenic Antigen-Specific T Cell Reactivity and Effector Memory T Cell Enrichment

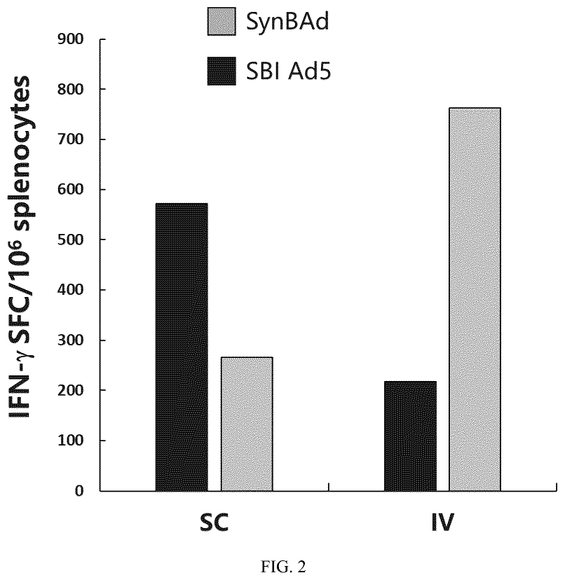

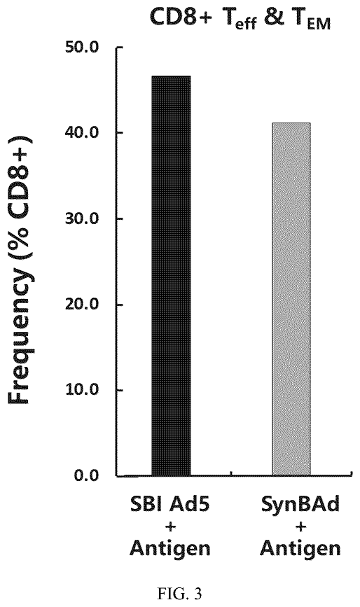

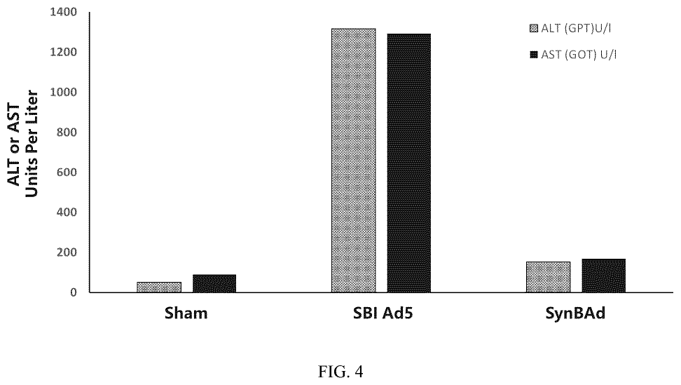

Both SBI Ad5 and SynBAd were found to produce significant IFN-γ SFC/106 splenocytes from the spleen as shown in FIG. 2. As described above, the animal subjects were administered the viruses at days 0 and 21, with collection and analysis on day 28. Surprisingly, the SBI Ad5 virus had higher levels compared to SynBAd when both were administered subcutaneously, while SynBAd produced significantly more when the constructs were administered intravenously. Both viruses elicited strong CD8 positive percentage of immune cells when administered with an antigen (ovalbumin, FIG. 3). Unexpectedly, the SynBAd virus demonstrates improved liver safety as compared to SBI Ad5, as the ALT or AST liver enzymes were not elevated with SynBAd administration, unlike SBI Ad5 where the liver enzyme levels increased significantly (FIG. 4). Thus, the SynBAd construct unexpectedly does not have a significant negative impact on the liver.

### Example 4: SynBAd Stimulates Robust Th1-Dominant, Antigen-Specific T Cell Responses

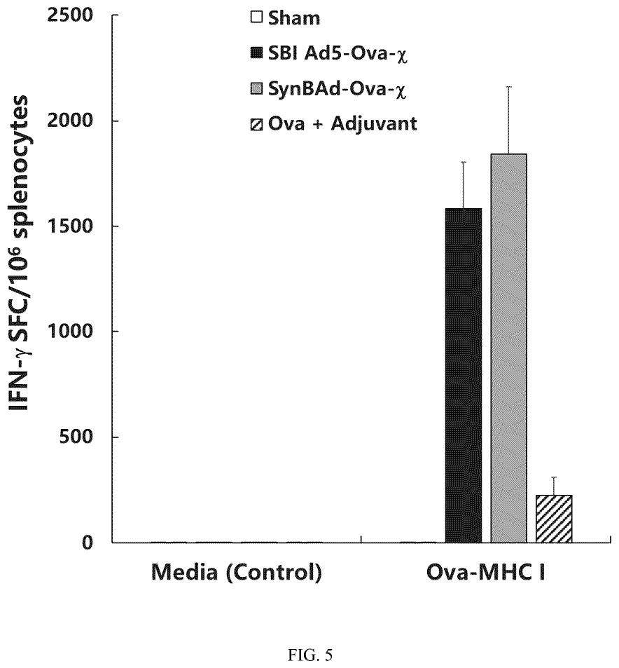

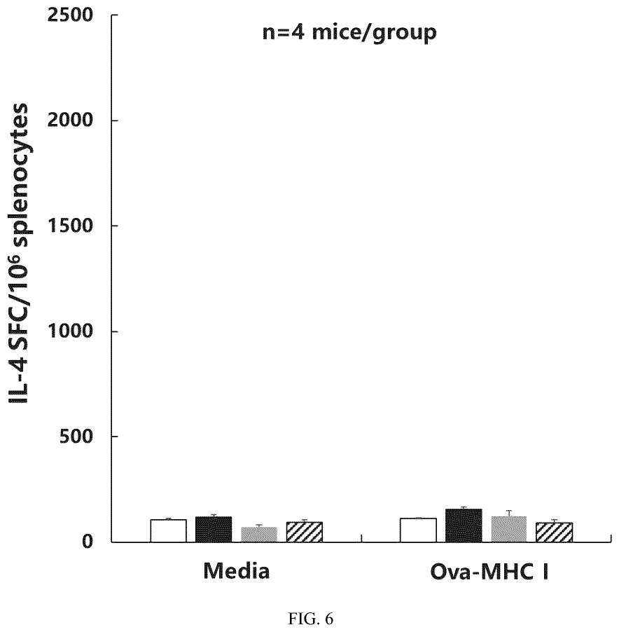

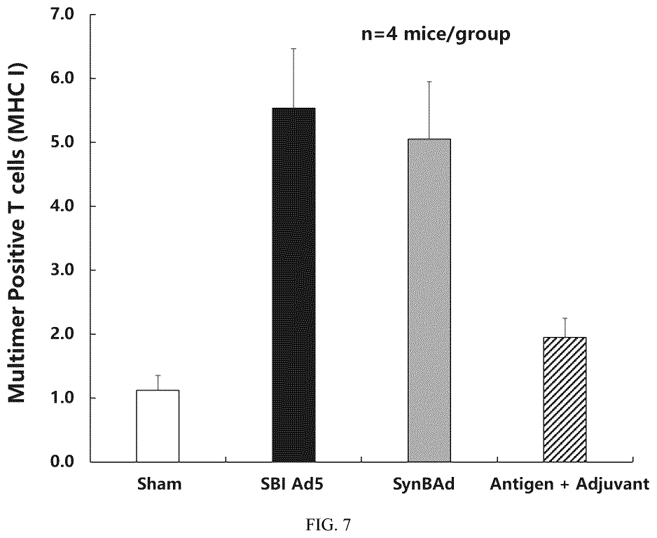

Using four groups: sham control, SBI Ad5 with ovalbumin (OVA) antigen, SynBAd with ovalbumin, and ovalbumin plus an adjuvant, animal administration and tissue collection was as described as above, but with only subcutaneous injections. FIG. 5 illustrates that SynBAd group show and substantial IFN-γ SFC/106 splenocytes. FIG. 6 illustrates that this effect is specific, as there was no corresponding IL-4 activity using the same 4 groups. Further, FIG. 7 illustrates that both the SBI Ad5 and SynBAd groups had similar levels of multimer positive T cell (MHCI) compared to the sham control and antigen plus adjuvant group.

In conclusion, the data in Examples 2-4 show that the SynBAd elicits robust antigen-specific T cell reactivity and enrichment, but with the surprising and unexpected result that the SynBAd construct has a stronger tropism (preference) for the spleen over the liver and the SynBAd construct has no apparent liver impact, with liver enzymes in the normal range following administration, which was the opposite of the construct with the wild-type hexon polypeptide.

### Immunology Assay Methods

Isolation of single cell suspensions: Organs of interest were harvest from immunized mice on Day 29. Single cell suspensions were isolated from spleen and liver by mechanical disruption and differential centrifugation.

Flow cytometry assays: Immune cell populations were identified using flow cytometry and antibodies directed against CD3, CD4, CD8, and NK1.1. Memory and effector T cell populations were identified with antibodies directed against CD44 and CD62L.

ELISpot: Functional responses from antigen-specific T cells were evaluated by ELISpot. In short, single cell suspensions were co-cultured with ovalbumin peptide overnight. Cytokine (IFN-γ and IL-4) secreting cells were enumerated using cytokine-specific antibodies.

### Example 5: Treatment of HBV Induced Cancer

A recombinant Ad comprising a transgene encoding a HBV polytope produced according to Example 1 comprising a hexon polypeptide of SEQ ID NO: 18, or a variant as provided herein, is administered to a patient with HBV induced liver cancer. The recombinant Ad transduces the liver with increased transduction efficiency and the cancer is treated.

What is claimed is:

1. A recombinant adenovirus (Ad), wherein capsid hexon polypeptides of an Ad strain Ad5 comprise at least one capsid hexon hypervariable region (HVR) polypeptide from Ad strain Ad57.

2. The recombinant Ad of claim 1, wherein the capsid hexon polypeptides of the Ad strain Ad5 comprises a variant of a polypeptide comprising the amino acid sequence of SEQ ID NO: 1, wherein the variant comprises:
i) an amino acid sequence of SEQ ID NO: 4, SEQ ID NO: 8, SEQ ID NO: 10, SEQ ID NO: 12, SEQ ID NO: 14, or SEQ ID NO: 16, or any combination thereof;
ii) one or more insertions of:
a polypeptide of EQ (Glu-Gln) between positions 156 and 157 of SEQ ID NO:1,
a polypeptide comprising amino acid sequence SEQ ID NO: 5 between positions 187 and 188 of SEQ ID NO: 1,
a polypeptide of TT (Thr-Thr) between positions 438 and 439 of SEQ ID NO: 1, or
a polypeptide of GATT (Gly-Ala-Thr-Thr; SEQ ID NO: 6) between positions 438 and 439 of SEQ ID NO: 1;
iii) one or more deletions of:
K252 of SEQ ID NO: 1,
polypeptide Q435E436 of SEQ ID NO: 1, or
E445 of SEQ ID NO: 1; or
any combination of replacements, insertions, and deletions of the foregoing.

3. The recombinant Ad of claim 1 or 2, wherein the hexon polypeptide of Ad strain Ad5 comprising the amino acid sequence of AATALEINLE (SEQ ID NO: 3) has SEQ ID NO: 3 replaced by an amino acid sequence of DDTQVQVAAE (SEQ ID NO: 4).

4. The recombinant Ad of any one of claims 1-3, wherein the hexon polypeptide of Ad strain Ad5 comprises the polypeptide of EQ between residue E (Glu) at position 156 and residue V (Val) at position 157 of SEQ ID NO: 1.

5. The recombinant Ad of any one of claims 1-4, wherein the hexon polypeptide of Ad strain Ad5 does not comprise an insertion, deletion, or substitution at, or between, residue T (Thr) at position 166 and residue F (Phe) at position 169 of SEQ ID NO: 1.

6. The recombinant Ad of any one of claims 1-5, wherein the hexon polypeptide of Ad strain Ad5 comprises an insertion of a polypeptide comprising the amino acid sequence of TNGAA (SEQ ID NO: 5) between residue G (Gly) at position 187 and residue V (Val) at position 188 of SEQ ID NO: 1.

7. The recombinant Ad of any one of claims 1-6, wherein the hexon polypeptide of Ad strain Ad5 does not comprise an insertion, deletion, or substitution at or between residue H (His) at position 218 and residue A (Ala) at position 220 of SEQ ID NO: 1.

8. The recombinant Ad of any one of claims 1-7, wherein the hexon polypeptide of Ad strain Ad5 comprises a deletion of residue K (Lys) at position 252 of SEQ ID NO: 1.

9. The recombinant Ad of any one of claims 1-8, wherein the hexon polypeptide of Ad strain Ad5 does not comprise an insertion, deletion, or substitution at or between residue F (Phe) at position 267 and residue S (Ser) at position 268 of SEQ ID NO: 1.

10. The recombinant Ad of any one of claims 1-9, wherein the hexon polypeptide of Ad strain Ad5 does not comprise an insertion, deletion, or substitution at or between residue G (Gly) at position 434 and residue Q (Gln) at position 435 of SEQ ID NO: 1.

11. The recombinant Ad of any one of claims 1-10, wherein the hexon polypeptide of Ad strain Ad5 comprises a deletion of residues QE at positions 435 and 436 of SEQ ID NO: 1.

12. The recombinant Ad of any one of claims 1-11, wherein the hexon polypeptide of Ad strain Ad5 comprises an insertion of a polypeptide comprising the sequence of TT between residue G at position 438 and residue W (Trp) at position 439 of SEQ ID NO: 1.

13. The recombinant Ad of any one of claims 1-12, wherein the hexon polypeptide of Ad strain Ad5 comprises an insertion of a polypeptide comprising the amino acid sequence of GATT (SEQ ID NO: 6) between residue G at position 438 and residue W at position 439 of SEQ ID NO: 1.

14. The recombinant Ad of any one of claims 1-12, wherein the hexon polypeptide of Ad strain Ad5 comprises a deletion of residue E at position 445 of SEQ ID NO: 1.

15. The recombinant Ad of any one of claims 1-14, wherein the hexon polypeptide of Ad strain Ad5 comprising the amino acid sequence of SEQ ID NO: 7 has the amino acid sequence of SEQ ID NO: 7 replaced by the amino acid sequence of SEQ ID NO: 8.

16. The recombinant Ad of any one of claims 1-15, wherein the hexon polypeptide of Ad strain Ad5 comprising the amino acid sequence of SEQ ID NO: 9 has the amino acid sequence of SEQ ID NO: 9 replaced by the amino acid sequence of SEQ ID NO: 10.

17. The recombinant Ad of any one of claims 1-16, wherein the hexon polypeptide of Ad strain Ad5 comprising the amino acid sequence of SEQ ID NO: 11 has the amino acid sequence of SEQ ID NO: 11 replaced by the amino acid sequence of SEQ ID NO: 12.

18. The recombinant Ad of any one of claims 1-17, wherein the hexon polypeptide of Ad strain Ad5 comprising the amino acid sequence of SEQ ID NO: 13 has the amino acid sequence of SEQ ID NO: 13 replaced by the amino acid sequence of SEQ ID NO: 14.

19. The recombinant Ad of any one of claims 1-18, wherein the hexon polypeptide of Ad strain Ad5 comprising the amino acid sequence of SEQ ID NO: 15 has the amino acid sequence of SEQ ID NO: 15 replaced by the amino acid sequence of SEQ ID NO: 16.

20. The recombinant Ad of any one of claims 1-19, wherein the hexon polypeptide of Ad strain Ad5 comprises a variant of the amino acid sequence of SEQ ID NO: 2 or SEQ ID NO: 18, wherein the variant comprises an aspartic acid (D) residue at position 864 and/or an arginine (R) residue at position 937 as compared to SEQ ID NO: 2 or SEQ ID NO: 18.

21. The recombinant Ad of claim 1, wherein the hexon polypeptide comprises an amino acid sequence of SEQ ID NO: 18.

22. The recombinant Ad of any one of claims 1-20, wherein the hexon polypeptide comprises a variant of a polypeptide comprising the amino acid sequence of SEQ ID NO: 18.

23. The recombinant Ad of claim 22, wherein variant hexon polypeptide comprises an amino acid sequence that is at least 75, 80, 85, 90, 91, 92, 93, 94, 95, 98, 97, 98, or 99% identical to the amino acid sequence of SEQ ID NO: 18.

24. The recombinant Ad of claim 22, wherein the variant hexon polypeptide comprises an amino acid sequence that is at least 75%, 80%, 85%, 90%, 91%, 92%, 93%, 94%, 95%, 96%, 97%, 98%, or 99% identical to the amino acid sequence of SEQ ID NO: 18, provided that the variant comprises:
the amino acid sequence of SEQ ID NO: 4, SEQ ID NO: 8, SEQ ID NO: 10, SEQ ID NO: 12, SEQ ID NO: 14, and SEQ ID NO: 16; or
the variant comprises an aspartic acid (D) residue at position 864 and/or an arginine (R) residue at position 937.

25. The recombinant Ad of claim 22, wherein the variant hexon polypeptide comprises 1, 2, 3, 4, 5, 6, 7, 8, 9, or 10 mutations (e.g., substitutions, insertions, or deletions), as compared to the amino acid sequence of SEQ ID NO: 18, provided that the variant comprises:
the amino acid sequence of SEQ ID NO: 4, SEQ ID NO: 8, SEQ ID NO: 10, SEQ ID NO: 12, SEQ ID NO: 14, and SEQ ID NO: 16; or
the variant comprises an aspartic acid (D) residue at position 864 and/or an arginine (R) residue at position 937.

26. The recombinant Ad of any one of claims 22-25, wherein the variant hexon polypeptide has increased in transduction in the spleen as compared to the liver.

27. The recombinant Ad of any one of claims 22-25, wherein the variant hexon polypeptide has increased in transduction in the spleen as compared to the liver as compared to a recombinant Ad comprising the hexon polypeptide having the amino acid sequence of SEQ ID NO: 1.

28. The recombinant Ad of any one of claims 22-25, wherein the variant hexon polypeptide does not increase liver enzymes when administered to a subject as compared to a recombinant Ad comprising the hexon polypeptide having the amino acid sequence of SEQ ID NO: 1

29. A recombinant Ad of any one of claims 1-28 further comprising a polynucleotide encoding a heterologous protein.

30. The recombinant Ad of claim 29, wherein the heterologous protein is a cytokine, an immunoglobulin, an immunomodulatory protein, a viral protein, a patient specific neoepitope, or any combination thereof.

31. The recombinant Ad of claim 30, wherein the viral protein is from HBV, HPV, EBV, CMV, HTLV, Polyoma, or any combination thereof.

32. The recombinant Ad of claim 30 or 31, wherein the immunomodulatory protein is from the B7 family.

33. The recombinant Ad of claim 32, wherein the immunomodulatory protein is B7.1 or B7.2.

34. The recombinant Ad of claims 32 and 33, wherein the immunomodulatory protein from the B7 family is fused to a domain from an immunoglobulin protein.

35. The recombinant Ad of claims 30-34, wherein the patient specific neoepitope is a tumor-specific antigen derived from somatic tumor mutation from a patient.

36. The recombinant Ad of any one of claims 1-35, further comprising a targeting moiety.

37. The recombinant Ad of claim 36, wherein the targeting moiety is a moiety that targets the Ad to carbohydrates, cell membranes and cells selected from muscle cells, tumors, cancer cells, kidney cells, liver cells, or mucosal cells.

38. A recombinant Ad of any one of claims 1-37, wherein the adenovirus is replication competent or is a conditionally-replicating Adenovirus (CRAd) that comprises a modified E1A gene encoding an E1A polypeptide, wherein the CRAd exhibits amino acid substitutions in the E1A polypeptide relative to wild-type E1A polypeptide of an Ad strain.

39. A nucleic acid molecule encoding capsid hexon polypeptides of an Ad strain Ad5 comprising at least one capsid hexon HVR polypeptide from Ad strain Ad57.

40. The nucleic acid molecule of claim 39, wherein the capsid hexon polypeptides of the Ad strain Ad5 comprises a variant of a polypeptide comprising the amino acid sequence of SEQ ID NO: 1, wherein the variant comprises:
i) an amino acid sequence of SEQ ID NO: 4, SEQ ID NO: 8, SEQ ID NO: 10, SEQ ID NO: 12, SEQ ID NO: 14, or SEQ ID NO: 16, or any combination thereof;
ii) one or more insertions of:
a polypeptide of EQ (Glu-Gln) between positions 156 and 157 of SEQ ID NO:1,
a polypeptide comprising amino acid sequence SEQ ID NO: 5 between positions 187 and 188 of SEQ ID NO: 1,
a polypeptide of TT (Thr-Thr) between positions 438 and 439 of SEQ ID NO: 1, or
a polypeptide of GATT (Gly-Ala-Thr-Thr; SEQ ID NO: 6) between positions 438 and 439 of SEQ ID NO: 1;
iii) one or more deletions of:
K252 of SEQ ID NO: 1,
polypeptide Q435E436 of SEQ ID NO: 1, or
E445 of SEQ ID NO: 1; or
any combination of replacements, insertions, and deletions of the foregoing.

41. The nucleic acid molecule of claim 39 or 40, wherein the nucleic acid molecule comprises a sequence of SEQ ID NO: 17.

42. The nucleic acid molecule of any one of claims 39-41, wherein the nucleic acid molecule encodes for a variant of a hexon polypeptide comprising the amino acid sequence of SEQ ID NO: 18.

43. The nucleic acid molecule of claim 42, wherein variant hexon polypeptide comprises an amino acid sequence that is at least 75, 80, 85, 90, 91, 92, 93, 94, 95, 96, 97, 98, or 99% identical to the amino acid sequence of SEQ ID NO: 18.

44. The nucleic acid molecule of claim 42, wherein the variant hexon polypeptide comprises an amino acid sequence that is at least 75%, 80%, 85%, 90%, 91%, 92%, 93%, 94%, 95%, 96%, 97%, 98%, or 99% identical to the amino acid sequence of SEQ ID NO: 18, provided that the variant comprises:
the amino acid sequence of SEQ ID NO: 4, SEQ ID NO: 8, SEQ ID NO: 10, SEQ ID NO: 12, SEQ ID NO: 14, and SEQ ID NO: 16; or
the variant comprises an aspartic acid (D) residue at position 864 and/or an arginine (R) residue at position 937.

45. The nucleic acid molecule of claim 42, wherein the variant hexon polypeptide comprises 1, 2, 3, 4, 5, 6, 7, 8, 9, or 10 mutations (e.g., substitutions, insertions, or deletions), as compared to the amino acid sequence of SEQ ID NO: 18, provided that the variant comprises:
the amino acid sequence of SEQ ID NO: 4, SEQ ID NO: 8, SEQ ID NO: 10, SEQ ID NO: 12, SEQ ID NO: 14, and SEQ ID NO: 16; or
the variant comprises an aspartic acid (D) residue at position 864 and/or an arginine (R) residue at position 937.

46. The nucleic acid molecule of any one of claims 42-45, wherein the variant hexon polypeptide has increased in transduction in the spleen as compared to the liver.

47. The nucleic acid molecule of any one of claims 42-45, wherein the variant hexon polypeptide has increased in transduction in the spleen as compared to the liver as compared to a recombinant Ad comprising the hexon polypeptide having the amino acid sequence of SEQ ID NO: 1.

48. The nucleic acid molecule of any one of claims 42-45, wherein the variant hexon polypeptide does not increase liver enzymes when administered to a subject as compared to a recombinant Ad comprising the hexon polypeptide having the amino acid sequence of SEQ ID NO: 1

49. A vector comprising the nucleic acid molecule of any one of any one of claims 39-49.

50. The vector of claim 49, wherein the vector is an Ad vector comprising a transgene encoding, for example, a heterologous polypeptide.

51. The vector of claim 50, wherein the Ad vector further comprises at least one of an E1 deletion and an E3 deletion.

52. A recombinant cell comprising the vector of any one of claims 49-51.

53. A method of producing a vector, comprising; (a) growing the recombinant cell of claim 52 under conditions for production of the vector; and (b) isolating the vector from the recombinant cell.

54. An immunogenic composition comprising the vector of any one of claims 49-51.

55. A method of inducing an immune response in a subject in need thereof, comprising administering to the subject the immunogenic composition of claim 54.

56. The method of claim 55, wherein the immune response is induced without significantly increasing liver enzymes in the subject.

57. The method of claim 56, wherein the immune response is induced without significantly increasing liver enzymes in the subject as compared to a vector comprising a hexon polypeptide having the amino acid sequence of SEQ ID NO: 1.

58. The method of any one of claims 55-57, wherein the composition is administered subcutaneously or intravenously.

59. A recombinant Ad strain Ad5 hexon polypeptide comprising at least one capsid hexon hypervariable region (HVR) polypeptide from Ad strain Ad57.

60. The recombinant hexon polypeptide of claim 59, wherein the capsid hexon polypeptides of the Ad strain Ad5 comprises a variant of a polypeptide comprising the amino acid sequence of SEQ ID NO: 1, wherein the variant comprises:
i) an amino acid sequence of SEQ ID NO: 4, SEQ ID NO: 8, SEQ ID NO: 10, SEQ ID NO: 12, SEQ ID NO: 14, or SEQ ID NO: 16, or any combination thereof;
ii) one or more insertions of:
a polypeptide of EQ (Glu-Gln) between positions 156 and 157 of SEQ ID NO:1,
a polypeptide comprising amino acid sequence SEQ ID NO: 5 between positions 187 and 188 of SEQ ID NO: 1,
a polypeptide of TT (Thr-Thr) between positions 438 and 439 of SEQ ID NO: 1, or
a polypeptide of GATT (Gly-Ala-Thr-Thr; SEQ ID NO: 6) between positions 438 and 439 of SEQ ID NO: 1;
iii) one or more deletions of:
K252 of SEQ ID NO: 1,
polypeptide Q435E436 of SEQ ID NO: 1, or
E445 of SEQ ID NO: 1; or
any combination of replacements, insertions, and deletions of the foregoing.

61. The recombinant hexon polypeptide of claim 59, wherein the hexon polypeptide comprises an amino acid sequence of SEQ ID NO: 18.

62. The recombinant hexon polypeptide of any one of claims 59-61, wherein the hexon polypeptide comprises a variant of a hexon polypeptide comprising the amino acid sequence of SEQ ID NO: 18.

63. The recombinant hexon polypeptide of claim 62, wherein the variant hexon polypeptide comprises an amino acid sequence that is at least 75, 80, 85, 90, 91, 92, 93, 94, 95, 95, 96, 98, or 99% identical to the amino acid sequence of SEQ ID NO: 18.

64. The recombinant hexon polypeptide of claim 62, wherein the variant hexon polypeptide comprises a variant of a polypeptide comprising an amino acid sequence that is at least 75%, 80%, 85%, 90%, 91%, 92%, 93%, 94%, 95%, 96%, 97%, 98%, or 99% identical to the amino acid sequence of SEQ ID NO: 18, provided that the variant comprises:
the amino acid sequence of SEQ ID NO: 4, SEQ ID NO: 8, SEQ ID NO: 10, SEQ ID NO: 12, SEQ ID NO: 14, and SEQ ID NO: 16; or
the variant comprises an aspartic acid (D) residue at position 864 and/or an arginine (R) residue at position 937.

65. The recombinant hexon polypeptide of claim 62, wherein the variant hexon polypeptide comprises 1, 2, 3, 4, 5, 6, 7, 8, 9, or 10 mutations (e.g., substitutions, insertions, or deletions), as compared to the amino acid sequence of SEQ ID NO: 18, provided that the variant comprises:
the amino acid sequence of SEQ ID NO: 4, SEQ ID NO: 8, SEQ ID NO: 10, SEQ ID NO: 12, SEQ ID NO: 14, and SEQ ID NO: 16; or
the variant comprises an aspartic acid (D) residue at position 864 and/or an arginine (R) residue at position 937.

66. A virus particle comprising the hexon polypeptide of any one of claims 59-61.


SEQUENCE LISTING (18 sequences)

SEQ ID NO: 1 - protein, 952 aa, unidentified
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 2 - protein, 959 aa, unidentified
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNSCEWDEDDTQVQVAAEDDQDDDEEEEQLPQQRNGKKTHVYAQAPFAGEAINKNGLQIGTNGAATEGNKEIYADKTYQPEPQIGESQWNEAESSVAGGRVLKKTTPMKPCYGSYARPTNSNGGQGVMVEQNGKLESQVEMQFFSTSVNAMNEANAIQPKLVLYSEDVNMETPDTHLSYKPGKSDDNSKAMLGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGIGVTDTYQAIKATNGNGGATTWAQDNTFAERNEIGVGNNFAMEINLNANLWRNFLYSNIALYLPDKLKYNPTNVEISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVNSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 3 - protein, 9 aa, synthetic construct
AATALEINL

SEQ ID NO: 4 - protein, 9 aa, synthetic construct
DDTQVQVAA

SEQ ID NO: 5 - protein, 5 aa, synthetic construct
TNGAA

SEQ ID NO: 6 - protein, 4 aa, synthetic construct
GATT

SEQ ID NO: 7 - protein, 122 aa, synthetic construct
PCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQ

SEQ ID NO: 8 - protein, 128 aa, synthetic construct
SCEWDEDDTQVQVAAEDDQDDDEEEEQLPQQRNGKKTHVYAQAPFAGEAINKNGLQIGTNGAATEGNKEIYADKTYQPEPQIGESQWNEAESSVAGGRVLKKTTPMKPCYGSYARPTNSNGGQGVMVE

SEQ ID NO: 9 - protein, 46 aa, synthetic construct
TEATAGNGDNLTPKVVLYSEDVDIETPDTHISYMPTIKEGNSRELM

SEQ ID NO: 10 - protein, 46 aa, synthetic construct
SVNAMNEANAIQPKLVLYSEDVNMETPDTHLSYKPGKSDDNSKAML

SEQ ID NO: 11 - protein, 34 aa, synthetic construct
VINTETLTKVKPKTGQENGWEKDATEFSDKNEIR

SEQ ID NO: 12 - protein, 35 aa, synthetic construct
IGVTDTYQAIKATNGNGGATTWAQDNTFAERNEIG

SEQ ID NO: 13 - protein, 6 aa, synthetic construct
SPSNVK

SEQ ID NO: 14 - protein, 6 aa, synthetic construct
NPTNVE

SEQ ID NO: 15 - protein, 370 aa, synthetic construct
NPCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNT

SEQ ID NO: 16 - protein, 377 aa, synthetic construct
SCEWDEDDTQVQVAAEDDQDDDEEEEQLPQQRNGKKTHVYAQAPFAGEAINKNGLQIGTNGAATEGNKEIYADKTYQPEPQIGESQWNEAESSVAGGRVLKKTTPMKPCYGSYARPTNSNGGQGVMVEQNGKLESQVEMQFFSTSVNAMNEANAIQPKLVLYSEDVNMETPDTHLSYKPGKSDDNSKAMLGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGIGVTDTYQAIKATNGNGGATTWAQDNTFAERNEIGVGNNFAMEINLNANLWRNFLYSNIALYLPDKLKYNPTNVEISDNPNTY

SEQ ID NO: 17 - DNA, 2880 bp, synthetic construct
  source 1..2880: mol_type = other DNA
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaattcatgcgagtgggatgaagatgatactcaggtacaggtagcggctgaagacgatcaagacgacgacgaagaagaggaacaactacctcagcagagaaatggcaaaaaaactcacgtatatgctcaggcaccgtttgctggcgaagcaattaacaaaaacggcctgcagataggaactaacggtgcagccactgaaggaaataaggaaatttacgcagataaaacttatcaacctgaaccacaaataggagaatcacagtggaacgaagccgaatcgtccgtagcaggtggaagggttcttaaaaagactactcccatgaaaccatgctatggctcctatgccagacctaccaattctaacggaggtcagggcgttatggttgaacaaaatggtaaattggaaagtcaagtagaaatgcaatttttttcaacttctgtaaatgctatgaacgaggcaaacgctattcaacctaaactagtgttgtatagtgaagatgtaaatatggaaaccccagacactcatctttcttataagcctggaaaaagtgatgataattctaaggcaatgttgggtcaacaatctatgccaaacagacccaattacatagctttcagggacaattttattggcctaatgtattacaacagcactggtaacatgggtgttcttgctggtcaggcatcacagctaaatgctgtcgtagatttgcaagacagaaacacagagctgtcctaccaacttttgcttgattctattggtgatcgaaccagatacttttccatgtggaatcaggctgtagacagctacgatccagatgttagaattatcgagaaccatggaactgaggatgaattgccaaattattgttttcctcttggcggaattggggtgacggacacctatcaagctattaaggctacaaatggaaatggaggcgccactacctgggctcaggacaatacttttgcagaacgaaatgaaataggggtgggaaataactttgccatggaaattaacctgaatgccaacctatggagaaatttcctttactccaatattgcgctgtacctgccagacaagctaaaatacaaccccaccaatgtggaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 18 - protein, 959 aa, synthetic construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNSCEWDEDDTQVQVAAEDDQDDDEEEEQLPQQRNGKKTHVYAQAPFAGEAINKNGLQIGTNGAATEGNKEIYADKTYQPEPQIGESQWNEAESSVAGGRVLKKTTPMKPCYGSYARPTNSNGGQGVMVEQNGKLESQVEMQFFSTSVNAMNEANAIQPKLVLYSEDVNMETPDTHLSYKPGKSDDNSKAMLGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGIGVTDTYQAIKATNGNGGATTWAQDNTFAERNEIGVGNNFAMEINLNANLWRNFLYSNIALYLPDKLKYNPTNVEISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT